(19) United States
(12) Patent Application Publication
(10) Pub. No.: US 2005/0124673 A1
(43) Pub. Date: Jun. 9, 2005

(54) Methods of preparing compounds useful as protease inhibitors

(75) Inventors: David John Kucera, Del Mar, CA (US); Robert William Scott, San Diego, CA (US)

Correspondence Address: AGOURON PHARMACEUTICALS, INC., 10350 NORTH TORREY PINES ROAD, LA JOLLA, CA, 92037, US

(73) Assignee: Agouron Pharmaceuticals, Inc.

(21) Appl. No.: 11/003,952

(22) Filed: Dec. 3, 2004

Related U.S. Application Data
(60) Provisional application No. 60/527,477, filed on Dec. 4, 2003.

(51) Int. Cl. A61K 31/426; A61K 31/421; A61K 31/401

(52) U.S. Cl. 514/365; 514/374; 514/423; 548/200; 548/215; 548/530; 562/450; 560/142

(57) ABSTRACT

The invention relates to methods of preparing compounds of formula (I), 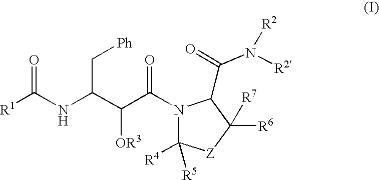 useful as inhibitors of the HIV protease enzyme. The present invention also relates to intermediate compounds useful in the preparation of compounds of formula (I).

This application claims priority under 35 U.S.C. § 119(e) to U.S. Provisional Application No. 60/527,477, filed Dec. 4, 2003, which is hereby incorporated by reference.

### BACKGROUND OF THE INVENTION

The present invention relates to methods of preparing, and intermediate compounds useful in the preparation of, inhibitors of the human immunodeficiency virus (HIV) protease.

Acquired Immune Deficiency Syndrome (AIDS) causes a gradual breakdown of the body's immune system as well as progressive deterioration of the central and peripheral nervous systems. Since its initial recognition in the early 1980's, AIDS has spread rapidly and has now reached epidemic proportions within a relatively limited segment of the population. Intensive research has led to the discovery of the responsible agent, human T-lymphotropic retrovirus III (HTLV-III), now more commonly referred to as HIV.

HIV is a member of the class of viruses known as retroviruses and is the etiologic agent of AIDS. The retroviral genome is composed of RNA, which is converted to DNA by reverse transcription. This retroviral DNA is then stably integrated into a host cell's chromosome and, employing the replicative processes of the host cells, produces new retroviral particles and advances the infection to other cells. HIV appears to have a particular affinity for the human T-4 lymphocyte cell, which plays a vital role in the body's immune system. HIV infection of these white blood cells depletes this white cell population. Eventually, the immune system is rendered inoperative and ineffective against various opportunistic diseases such as, among others, pneumocystic carini pneumonia, Kaposi's sarcoma, and cancer of the lymph system.

Although the exact mechanism of the formation and working of the HIV virus is not understood, identification of the virus has led to some progress in controlling the disease. For example, the drug azidothymidine (AZT) has been found effective for inhibiting the reverse transcription of the retroviral genome of the HIV virus, thus giving a measure of control, though not a cure, for patients afflicted with AIDS. The search continues for drugs that can cure or at least provide an improved measure of control of the deadly HIV virus and thus the treatment of AIDS and related diseases.

Retroviral replication routinely features post-translational processing of polyproteins. This processing is accomplished by virally encoded HIV protease enzyme. This yields mature polypeptides that will subsequently aid in the formation and function of infectious virus. If this molecular processing is stifled, then the normal production of HIV is terminated. Therefore, inhibitors of HIV protease may function as anti-HIV viral agents.

HIV protease is one of the translated products from the HIV structural protein pol 25 gene. This retroviral protease specifically cleaves other structural polypeptides at discrete sites to release these newly activated structural proteins and enzymes, thereby rendering the virion replication-competent. As such, inhibition of the HIV protease by potent compounds may prevent proviral integration of infected T-lymphocytes during the early phase of the HIV-1 life cycle, as well as inhibit viral proteolytic processing during its late stage. Additionally, the protease inhibitors may have the advantages of being more readily available, longer lived in virus, and less toxic than currently available drugs, possibly due to their specificity for the retroviral protease.

Methods for preparing compounds useful as HIV protease inhibitors have been described in, e.g., U.S. Pat. No. 5,962,640; U.S. Pat. No. 5,932,550; U.S. Pat. No. 6,222,043; U.S. Pat. No. 5,644,028; WO 02/100844, Australian Patent No. 705193; Canadian Patent Application No. 2,179,935; European Patent Application No. 0 751 145; Japanese Patent Application No. 100867489; Y. Hayahsi, et al., J. Org. Chem., 66, 5537-5544 (2001); K. Yoshimura, et al., Proc. Natl. Acad. Sci. USA, 96, 8675-8680 (1999); and, T. Mimoto, et al., J. Med. Chem., 42, 1789-1802 (1999). Thus, methods of preparing compounds useful as protease inhibitors have previously been known. However, these methods were linear and thus inefficient. The improved methods of the invention provide for convergent synthetic routes having maximized efficiency.

### SUMMARY OF THE INVENTION

The present invention relates to methods of preparing compounds of formula (I), or a salt or solvate thereof: 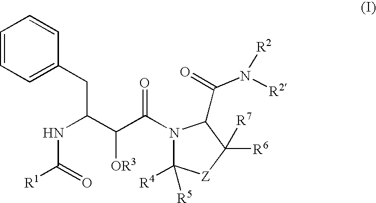
wherein:

R¹ is phenyl optionally substituted by at least one substituent independently chosen from C₁-C₆ alkyl, hydroxyl, C₁-C₆ alkylcarbonyloxy, C₆-C₁₀ arylcarbonyloxy, and heteroarylcarbonyloxy;
R² is C₂-C₆ alkenyl, C₁-C₆ alkyl optionally substituted with at least one halogen, or —(CR⁴R⁵)ₙR⁸;
n is an integer from 0 to 5;
R^{2′} is H or C₁-C₄ alkyl;
Z is S, O, SO, SO₂, CH₂, or CFH;
R³ is hydrogen or a hydroxyl protecting group;
each R⁴, R⁵, R⁶ and R⁷ are independently selected from H and C₁-C₆ alkyl; and
R⁸ is C₆-C₁₀ aryl optionally substituted at least one substituent selected from C₁-C₆ alkyl, hydroxyl, and halogen;
   comprising:
reacting a compound of formula (II), wherein Y¹ is hydroxyl or a leaving group and R¹ is as described for formula (I), with a compound of formula (III), or a salt or solvate thereof.

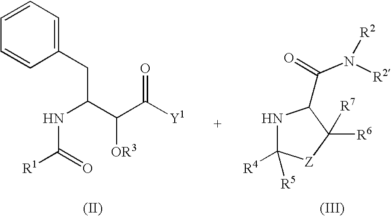

The present invention further comprises deprotecting the compound of formula (I) when R³ is a hydroxyl-protecting group to afford a compound of formula (I) wherein R³ is hydrogen.

The present invention also provides intermediate compounds that are useful for the preparation of compounds of formula (I).

The following describe further embodiments of the present invention.

In another aspect of the present invention are provided methods for preparing compounds of formula (I), 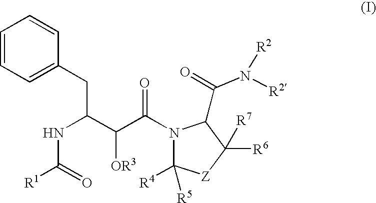
wherein:

R¹ is phenyl optionally substituted by at least one substituent independently chosen from C₁-C₆ alkyl, hydroxyl, C₁-C₆ alkylcarbonyloxy, C₆-C₁₀ arylcarbonyloxy, and heteroarylcarbonyloxy;
R² is C₂-C₆ alkenyl, C₁-C₆ alkyl optionally substituted with at least one halogen, or —(CH₂)ₙR⁸;
n is an integer from 0-5;
R² is H or C₁-C₄ alkyl;
Z is S, O, SO, SO₂, CH₂, or CFH;
R³ is hydrogen or a hydroxyl-protecting group;
R⁴, R⁵, R⁶ and R⁷ are independently selected from H and C₁-C₆ alkyl; and

R⁸ is C₆-C₁₀ aryl optionally substituted at least one substituent selected from C₁-C₆ alkyl, hydroxyl, and halogen;

comprising:

reacting a compound of formula (II), wherein Y¹ is hydroxyl or a leaving group, with a compound of formula (III), or a salt or solvate thereof,

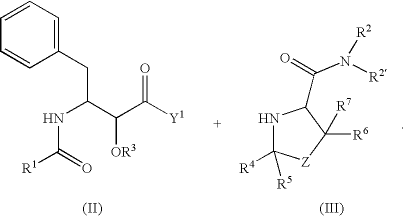

In another aspect of the present invention are provided methods for the preparation of compounds of formula (I), comprising:
(i) reacting a compound of formula (IV), wherein Y¹ is hydroxy or —OP¹, wherein P¹ is a suitable protecting group, and R³ is hydrogen, C₁-C₄ alkyl, or a suitable hydroxyl protecting group, with a compound of formula (V), wherein Y² is a leaving group, to afford a compound of formula (II); 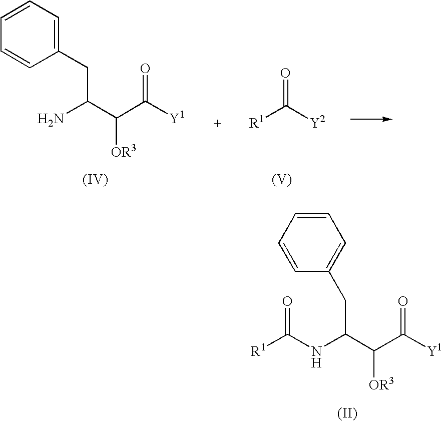
(ii) reacting the compound of formula (II) with a compound of formula (III), or a salt or solvate thereof, to afford a compound of formula (I); and 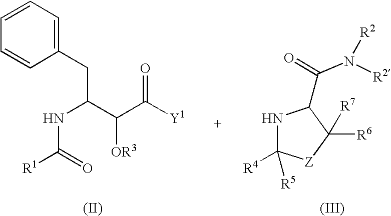
(iii) optionally deprotecting those compounds of formula (i) wherein R³ is a hydroxyl protecting group, to afford a compound of formula (I) wherein R³ is hydrogen.

In another aspect of the present invention are provided any of the methods described herein of preparing the compounds of the formula (I) wherein in the compound of (II) Y¹ is hydroxyl.

In still another aspect of the present invention are provided any of the methods described herein for the preparation of compounds of formula (I), wherein:
R¹ is phenyl optionally substituted by at least one substituent independently chosen from C₁-C₆ alkyl, hydroxyl, C₁-C₆ alkylcarbonyloxy, C₆-C₁₀ arylcarbonyloxy, and heteroarylcarbonyloxy;
R² is C₂-C₆ alkenyl, C₁-C₆ alkyl optionally substituted with at least one halogen, or —(CH₂)ₙR⁸;
n is 0, 1, 2, or 3;
R²′ is H;
Z is S, O, CH₂, or CFH;
R³ is hydrogen or a hydroxyl-protecting group;
R⁴ and R⁵ are hydrogen;
R⁶ and R⁷ are C₁-C₆ alkyl; and
R⁸ is C₆-C₁₀ optionally substituted at least one substituent selected from C₁-C₆ alkyl, hydroxyl, and halogen.

Yet another aspect of the present invention provides any of the methods described herein for the preparation of compounds of formula (I), wherein:
R¹ is phenyl optionally substituted by at least one substituent independently chosen from C₁-C₆ alkyl, hydroxyl, C₁-C₆ alkylcarbonyloxy, C₆-C₁₀ arylcarbonyloxy, and heteroarylcarbonyloxy;
R² is C₂-C₆ alkenyl, C₁-C₆ alkyl optionally substituted with at least one halogen, or —(CH₂)ₙR⁸;
n is 0, 1, 2, or 3;
R²′ is H;
Z is S;
R³ is hydrogen;
R⁴ and R⁵ are hydrogen;
R⁶ and R⁷ are methyl; and

R⁸ is phenyl optionally substituted at least one substituent selected from C₁-C₆ alkyl, hydroxyl, and halogen.

In yet another aspect of the present invention provides any of the methods described herein for the preparation of compounds of formula (I), wherein:
R¹ is phenyl optionally substituted by at least one substituent independently chosen from C₁-C₆ alkyl, hydroxyl, C₁-C₆ alkylcarbonyloxy, C₆-C₁₀ arylcarbonyloxy, and heteroarylcarbonyloxy;
R² is C₂-C₆ alkenyl, C₁-C₆ alkyl optionally substituted with at least one halogen, or —(CH₂)ₙR⁸;
n is 0, 1, 2, or 3;
R^{2′} is H;
Z is S;
R³ is a hydroxyl protecting group;
R⁴ and R⁵ are hydrogen;
R⁵ and R⁷ are methyl; and
R⁸ is phenyl optionally substituted at least one substituent selected from C₁-C₆ alkyl, hydroxyl, and halogen.

Another aspect of the present invention provides any of the methods described herein for the preparation of compounds of formula (I), wherein:
R¹ is phenyl optionally substituted by at least one substituent independently chosen from methyl, hydroxyl, and methylcarbonyloxy;
R² is C₂-C₆ alkenyl, C₁-C₆ alkyl optionally substituted with at least one halogen, or —CH₂R⁸;
R^{2′} is H;
Z is S;
R³ is hydrogen or a hydroxyl-protecting group;
R⁴ and R⁵ are hydrogen;
R⁶ and R⁷ are methyl; and
R⁸ is phenyl substituted with at least one methyl.

The present invention also provides any of the methods described herein for the preparation of compounds of formula (I), wherein:

R¹ is phenyl optionally substituted by at least one substituent independently chosen from methyl, hydroxyl, and methylcarbonyloxy;
R² is C₂-C₆ alkenyl;
R^{2′} is H;
Z is S;
R³ is hydrogen or a hydroxyl-protecting group;
R⁴ and R⁵ are hydrogen; and
R⁶ and R⁷ are methyl.

Also provided in the present invention are any of the methods described herein for the preparation of compounds of formula (1), wherein:
R¹ is phenyl substituted by methyl and hydroxyl;
R² is allyl;
R^{2′} is H;
Z is S;
R³ is hydrogen or methylcarbonyl;
R⁴ and R⁵ are hydrogen; and
R⁶ and R⁷ are methyl.

The present invention also provides any of the methods described herein for the preparation of compounds of formula (I), wherein:
R¹ is phenyl substituted with methyl and methylcarbonyloxy;
R² is allyl;
R^{2′} is H;
Z is S;
R³ is methylcarbonyl;
R⁴ and R⁵ are each H; and
R⁶ and R⁷ are methyl.
Also provided in the present invention are any of the methods described herein for the preparation of compounds of formula (I), wherein the compound of formula (I) is:

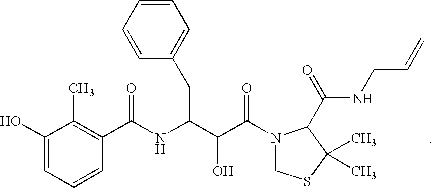

Still another aspect of the present invention provides methods for the preparation of compounds of formula (I-A), 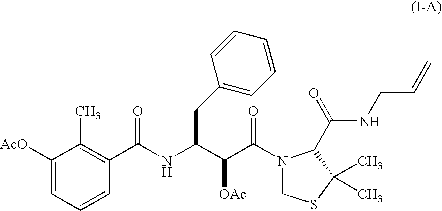
comprising:
reacting a compound of formula (II-A) with a compound of formula (III-A), or a salt or solvate thereof.

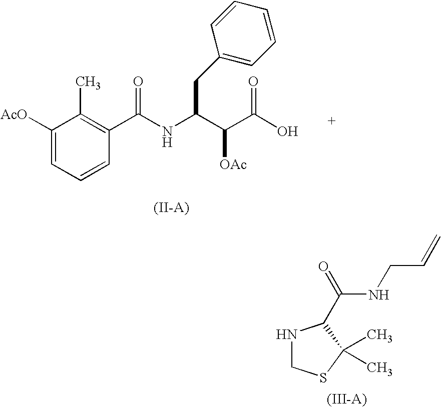

In still another aspect of the present invention are provided methods for the preparation of preparing compounds of formula (I-A), 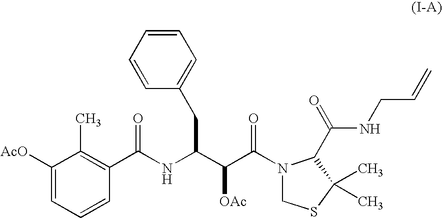
comprising:
(i) reacting a compound of formula (IV-A) with a compound of formula (V-A), 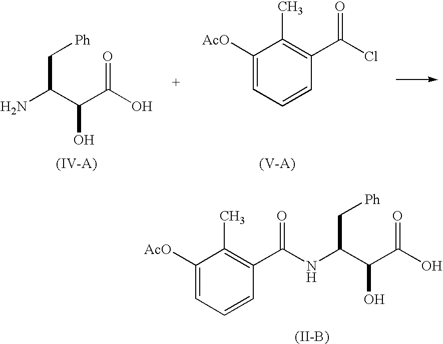
   to afford a compound of formula (II-B);
(ii) treating the compound of formula (II-B) with an acetylating agent to afford a compound of formula (I-A); and 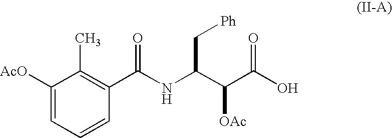
(iii) reacting the compound of formula (I-A) with a compound of formula (III-A).

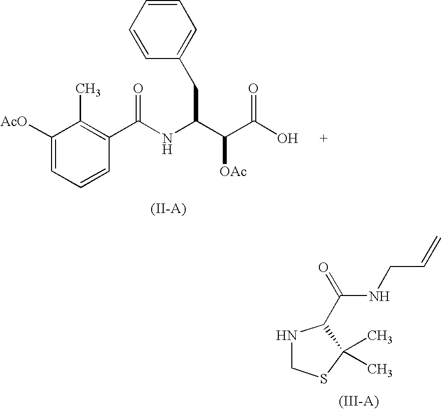

In yet another aspect of the present invention are provided methods for the preparation of compounds of formula (I-B), 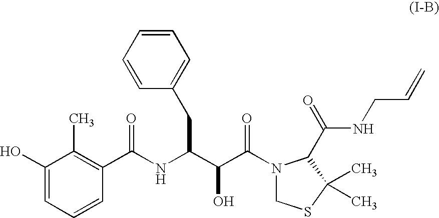
comprising:
(i) reacting a compound of formula (II-A) with a compound of formula (III-A), or a salt or solvate thereof, 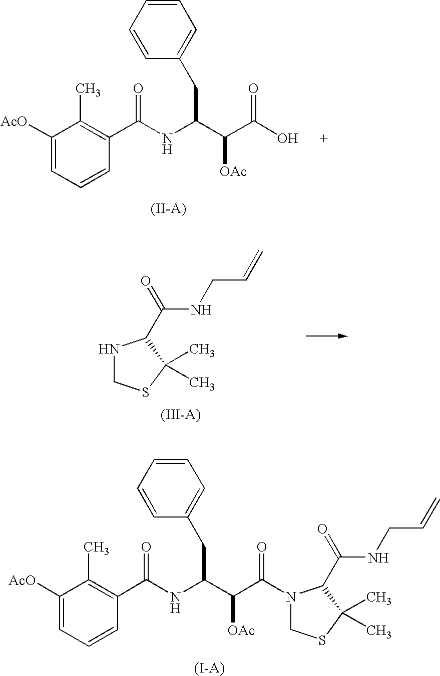
   to afford a compound of formula (I-A); and
(ii) deprotecting the compound of formula (I-A).

Another aspect of the present invention provides a method of preparing a compound of formula (I-B), 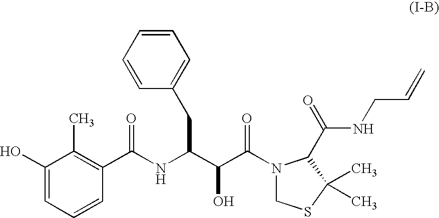
comprising:
(i) reacting a compound of formula (IV-A) with a compound of formula (V-A), 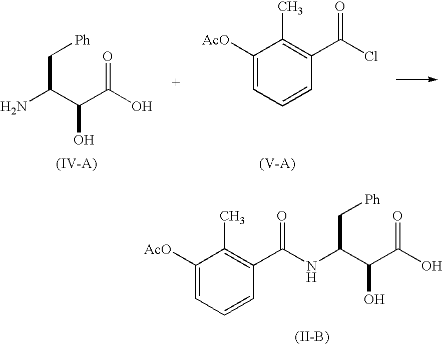
   to afford a compound of formula (II-B);
(ii) treating the compound of formula (II-B) with an acetylating agent to afford a compound of formula (II-A); and 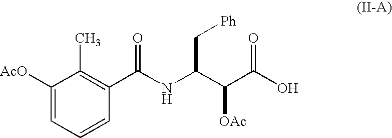
(iii) reacting the compound of formula (II-A) with a compound of formula (III-A), 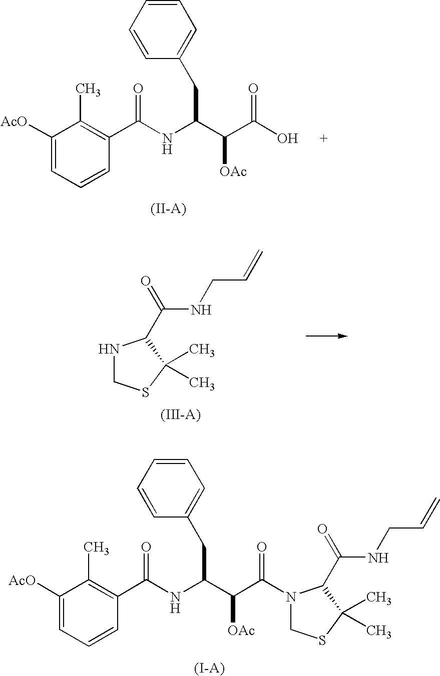
   to afford a compound of formula (I-A); and
(iv) deprotecting the compound of formula (I-A).

In another aspect of the present invention are provided any of the methods described herein for the preparation of compounds of formula (I), wherein:
R¹ is phenyl optionally substituted by at least one substituent independently chosen from methyl, hydroxyl, and methylcarbonyloxy;
R² is —CH₂R⁸;
R^{2′} is H;
Z is S;
R³ is hydrogen or a hydroxyl-protecting group;
R⁴ and R⁵ are hydrogen;
R⁶ and R⁷ are methyl; and
R⁸ is phenyl substituted with at least one methyl.

In yet another aspect of the present invention are provided any of the methods described herein for preparing compounds of formula (I), wherein:
R¹ is phenyl substituted by methyl and methylcarbonyloxy;
R² is —CH₂R⁸;
R^{2′} is H;
Z is S;
R³ is methylcarbonyl;
R⁴ and R⁵ are hydrogen;
R⁶ and R⁷ are methyl; and
R⁸ is phenyl substituted with at least one methyl.

In still a further aspect of the present invention are provided any of the methods described herein for the preparation of compounds of formula (I), wherein:
R¹ is phenyl substituted by methyl and hydroxyl;
R² is —CH₂R⁸;
R^{2′} is H;
Z is S;
R³ is hydrogen;
R⁴ and R⁵ are hydrogen;
R⁶ and R⁷ are methyl; and
R⁸ is phenyl substituted with at least one methyl.

Also provided in the present invention are any of the methods described herein for the preparation of compounds of formula (I), wherein the compound of formula (I) is:

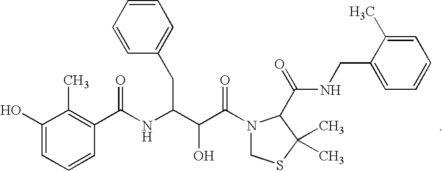

Yet another aspect of the present invention provides methods for the preparation of compounds of formula (I-C), 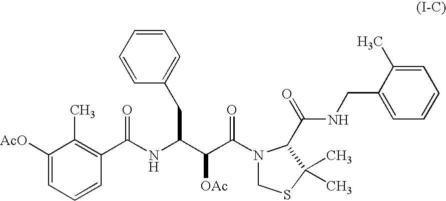
comprising:
reacting a compound of formula (II-A) with a compound of formula (III-B), or a salt or solvate thereof.

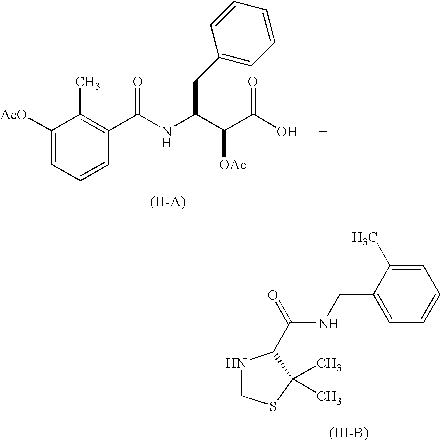

In still another aspect of the present invention are provided methods for the preparation of preparing compounds of formula (I-C), 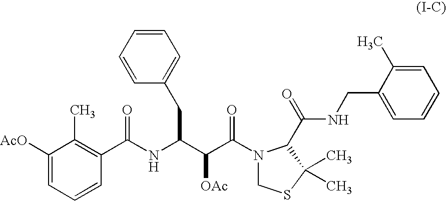
comprising:
(i) reacting a compound of formula (IV-A) with a compound of formula (V-A), 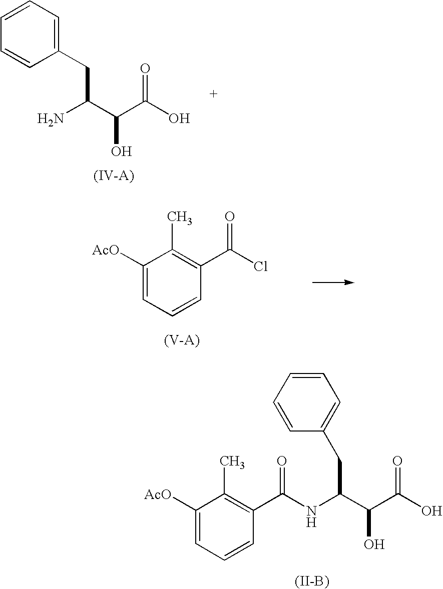
   to afford a compound of formula (II-B);
(ii) treating the compound of formula (II-B) with an acetylating agent to afford a compound of formula (II-A); and 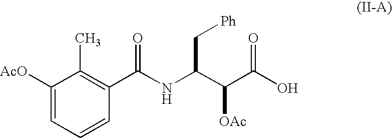
(iii) reacting the compound of formula (II-A) with a compound of formula (III-B),

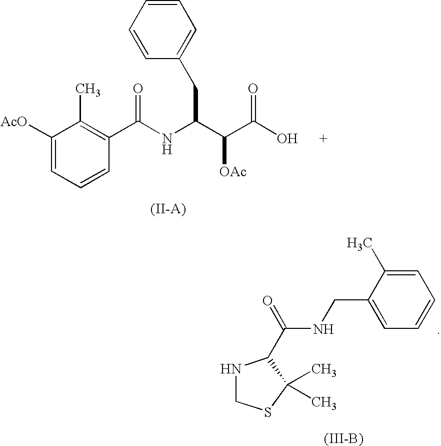

In yet another aspect of the present invention are provided methods for the preparation of compounds of formula (I-D), 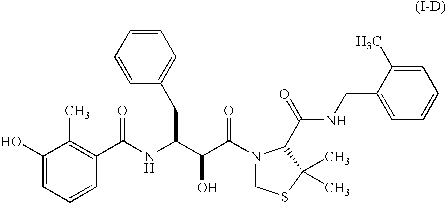
comprising:
(i) reacting a compound of formula (II-A) with a compound of formula (III-B), or a salt or solvate thereof, 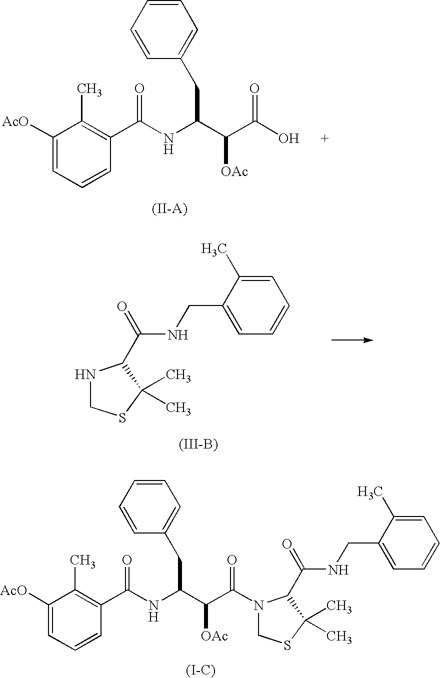
   to afford a compound of formula (I-C); and
(ii) deprotecting the compound of formula (I-C).

Another aspect of the present invention provides a method of preparing a compound of formula (I-D), 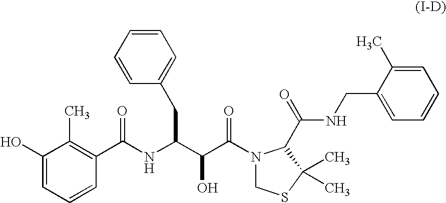
comprising:
(i) reacting a compound of formula (IV-A) with a compound of formula (V-A), 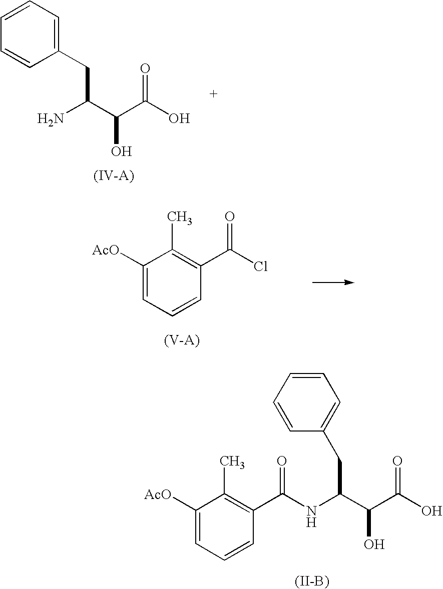
   to afford a compound of formula (II-B);
(ii) treating the compound of formula (II-B) with an acetylating agent to afford a compound of formula (II-A); and 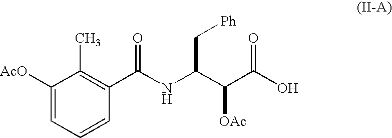
(iii) reacting the compound of formula (II-A) with a compound of formula (III-B), 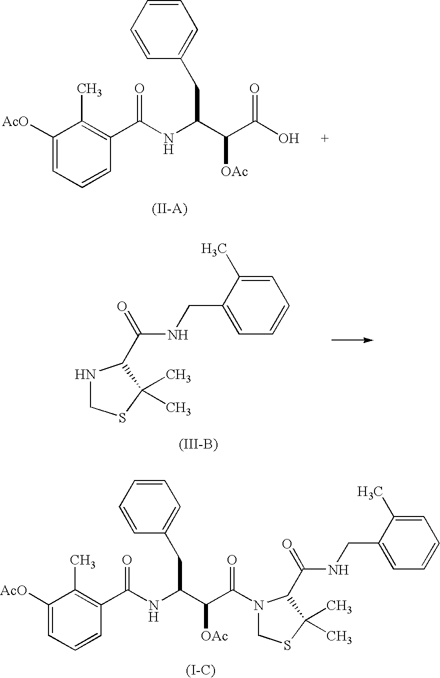
   to afford a compound of formula (I-C); and
(iv) deprotecting the compound of formula (I-C) to afford the compound of formula (I-D).

Another aspect of the present invention features compounds of formulae (I-A), (I-B), (II-A), (III-A), (III-B), (I-C), and (I-D): 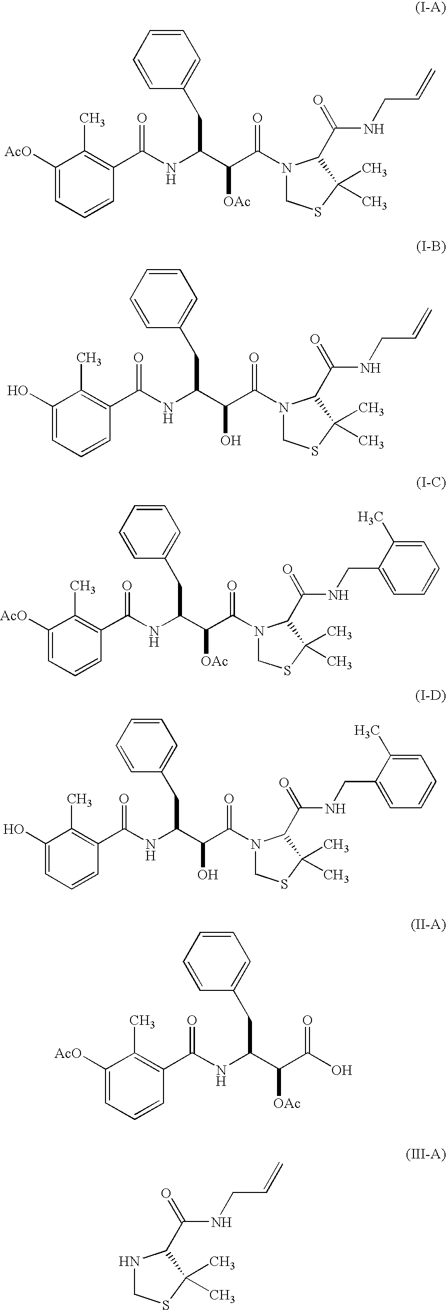 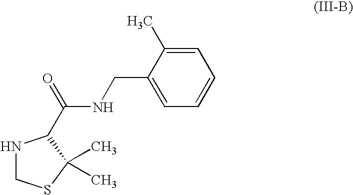
all of which are intermediates useful in the preparation of compounds of formula (I).

Another aspect of the present invention provides for the preparation of compounds of formula (II-A), 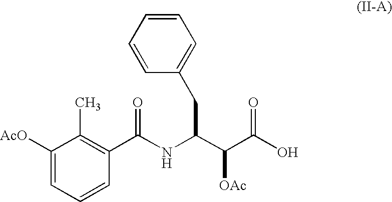
comprising treating a compound of formula (II-B) with an acetylating agent. In another aspect of the present invention, said acetylating agent is chosen from acetic anhydride and acetyl chloride.

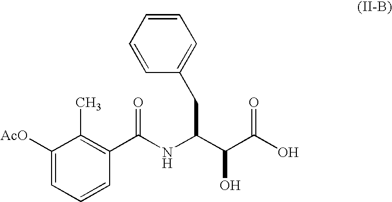

### DETAILED DESCRIPTION OF THE INVENTION

In accordance with a convention used in the art 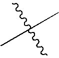
is used in structural formulas herein to depict the bond that is the point of attachment of the moiety or substituent to the core or backbone structure. When the phrase, “substituted with at least one substituent” is used herein, it is meant to indicate that the group in question may be substituted by at least one of the substituents chosen. The number of substituents a group in the compounds of the invention may have depends on the number of positions available for substitution. For example, an aryl ring in the compounds of the invention may contain from 1 to 5 additional substituents, depending on the degree of substitution present on the ring. The maximum number of substituents that a group in the compounds of the invention may have can be determined by those of ordinary skill in the art.

The term “reacting,” as used herein, refers to a chemical process or processes in which two or more reactants are allowed to come into contact with each other to effect a chemical change or transformation. For example, when reactant A and reactant B are allowed to come into contact with each other to afford a new chemical compound(s) C, A is said to have “reacted” with B to produce C.

The term “protecting,” as used herein, refers to a process in which a functional group in a chemical compound is selectively masked by a non-reactive functional group in order to allow a selective reaction(s) to occur elsewhere on said chemical compound. Such non-reactive functional groups are herein termed “protecting groups.” For example, the term “hydroxyl protecting group,” as used herein refers to those groups that are capable of selectively masking the reactivity of a hydroxyl (—OH) group. The term “suitable protecting group,” as used herein refers to those protecting groups that are useful in the preparation of the compounds of the present invention. Such groups are generally able to be selectively introduced and removed using mild reaction conditions that do not interfere with other portions of the subject compounds. Protecting groups that are suitable for use in the processes and methods of the present invention are known to those of ordinary skill in the art. The chemical properties of such protecting groups, methods for their introduction and their removal can be found, for example, in T. Greene and P. Wuts, Protective Groups in Organic Synthesis (3rd ed.), John Wiley & Sons, NY (1999). The terms “deprotecting,” “deprotected,” or “deprotect,” as used herein, are meant to refer to the process of removing a protecting group from a compound.

The term “leaving group,” as used herein refers to a chemical functional group that generally allows a nucleophilic substitution reaction to take place at the atom to which it is attached. For example, in acid chlorides of the formula Cl—C(O)R, wherein R is alkyl, aryl, or heterocyclic, the —Cl group is generally referred to as a leaving group because it allows nucleophilic substitution reactions to take place at the carbonyl carbon. Suitable leaving groups are known to those of ordinary skill in the art and can include halides, aromatic heterocycles, cyano, amino groups (generally under acidic conditions), ammonium groups, alkoxide groups, carbonate groups, formates, and hydroxy groups that have been activated by reaction with compounds such as carbodiimides. For example, suitable leaving groups can include, but are not limited to, chloride, bromide, iodide, cyano, imidazole, and hydroxy groups that have been allowed to react with a carbodiimide such as dicyclohexylcarbodiimide (optionally in the presence of an additive such as hydroxybenzotriazole) or a carbodiimide derivative.

The term “acetylating agent,” as used herein refers to chemical compounds that are useful for the introduction of an acetyl group, —C(O)CH₃, onto a hydroxyl group in the compounds of the invention. The symbol “Ac—,” as used in chemical structures herein, is meant to represent an acyl group in the compounds of the invention. Useful acetylating agents include, but are not limited to, acetic anhydride, acetyl chloride, acetyl bromide, and acetyl iodide. In addition, such acetylating agents can be prepared in situ by reaction of an appropriate combination of compounds, such as the reaction of acetyl chloride with sodium iodide in acetone to afford an intermediate acetyl iodide agent. The term “acetic anhydride,” as used herein is meant to represent a compound with the chemical formula CH₃C(O)OC(O)CH₃. In addition, the term “—OAc,” as used in the chemical structures herein, represents the group —OC(O)CH₃.

As used herein, the term “aliphatic” represents a saturated or unsaturated, straight- or branched-chain hydrocarbon, containing 1 to 10 carbon atoms which may be unsubstituted or substituted by one or more of the substituents described below. The term “aliphatic” is intended to encompass alkyl, alkenyl and alkynyl groups.

As used herein, the terms “C₁₋₁₀ alkyl” and “C₁-C₆ alkyl,” which may be used interchangeably throughout, represents a straight- or branched-chain saturated hydrocarbon, containing 1 to 6 carbon atoms which may be unsubstituted or substituted by one or more of the substituents described below. Similarly, the terms “C₁₋₄ alkyl” and “C₁-C₄ alkyl,” which may be used interchangeably throughout, represents a straight- or branched-chain saturated hydrocarbon, containing from 1 to 4 carbon atoms which may be unsubstituted or substituted by one or more of the substituents described below. Exemplary alkyl substituents include, but are not limited to methyl (Me), ethyl (Et), propyl, isopropyl, butyl, isobutyl, t-butyl, and the like.

The terms “C₂₋₆ alkenyl” and “C₂-C₆ alkenyl,” which may be used interchangeably throughout, represent a straight- or branched-chain hydrocarbon, containing one or more carbon-carbon double bonds and having 2 to 6 carbon atoms which may be unsubstituted or substituted by one or more of the substituents described below. Exemplary alkenyl substituents include, but are not limited to ethenyl, propenyl, butenyl, allyl, pentenyl and the like.

The terms “C₆₋₁₄ aryl” and “C₆-C₁₄ aryl”, which may be used interchangeably throughout, and as used herein, mean a group derived from an aromatic hydrocarbon containing from 6 to 14 carbon atoms. Examples of such groups include, but are not limited to, phenyl or naphthyl. The terms “Ph” and “phenyl,” as used herein, mean a —C₆H₅ group. The term “benzyl,” as used herein, means a —CH₂C₆H₅ group. The term “phenyl,” as used herein refers to a fully unsaturated 6-membered carbocyclic group. The symbol “Ph,” as used in the chemical structures herein, is meant to represent a phenyl or C₆H₅— group.

The term “heteroaryl,” as used herein refers to a group comprising an aromatic monovalent monocyclic, bicyclic, or tricyclic group, containing 5 to 18 ring atoms, including 1 to 5 heteroatoms selected from nitrogen, oxygen and sulfur, which may be unsubstituted or substituted by one or more of the substituents described below. As used herein, the term “heteroaryl” is also intended to encompass the N-oxide derivative (or N-oxide derivatives, if the heteroaryl group contains more than one nitrogen such that more than one N-oxide derivative may be formed) of the nitrogen-containing heteroaryl groups described herein. Illustrative examples of heteroaryl groups include, but are not limited to, thienyl, pyrrolyl, imidazolyl, pyrazolyl, furyl, isothiazolyl, furazanyl, isoxazolyl, thiazolyl, pyridyl, pyrazinyl, pyrimidinyl, pyridazinyl, triazinyl, benzo[b]thienyl, naphtho[2,3-b]thianthrenyl, isobenzofuranyl, chromenyl, xanthenyl, phenoxathienyl, indolizinyl, isoindolyl, indolyl, indazolyl, purinyl, isoquinolyl, quinolyl, phthalazinyl, naphthyridinyl, quinoxyalinyl, quinzolinyl, benzothiazolyl, benzimidazolyl, tetrahydroquinolinyl, cinnolinyl, pteridinyl, carbazolyl, beta-carbolinyl, phenanthridinyl, acridinyl, perimidinyl, phenanthrolinyl, phenazinyl, isothiazolyl, phenothiazinyl, and phenoxazinyl. Illustrative examples of N-oxide derivatives of heteroaryl groups include, but are not limited to, pyridyl N-oxide, pyrazinyl N-oxide, pyrimidinyl N-oxide, pyridazinyl N-oxide, triazinyl N-oxide, isoquinolyl N-oxide, and quinolyl N-oxide. Further examples of heteroaryl groups include the following moieties: 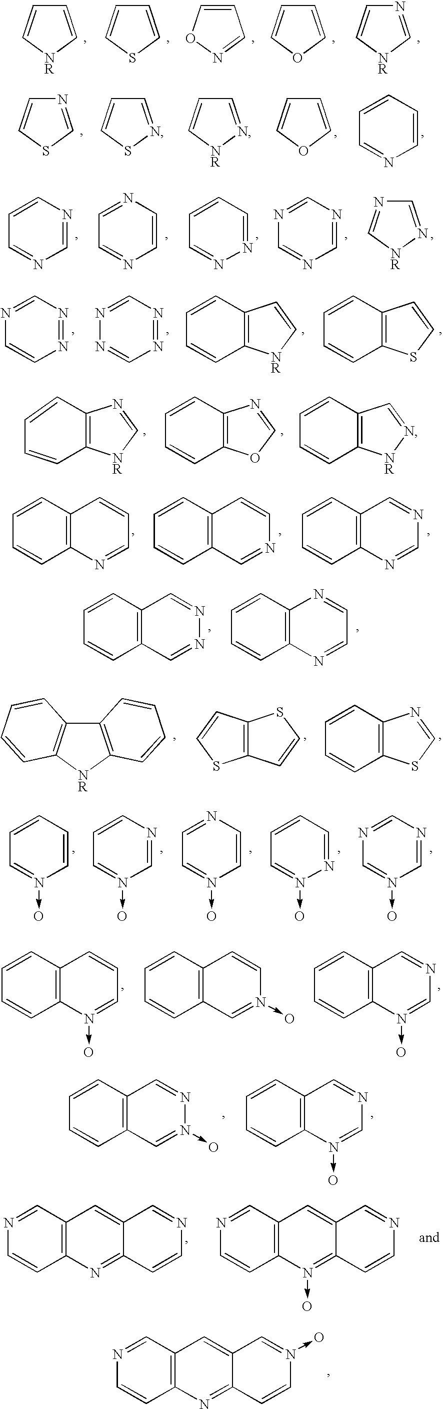
wherein R is H, alkyl, hydroxyl or represents a compound according to Formula I.

The terms “halogen” and “halo” represent chloro, fluoro, bromo or iodo substituents.

The terms “C₁₋₆ alkylcarbonyloxy” and “C₁-C₆ alkylcarbonyloxy,” which may be used interchangeably throughout, and as used herein, refers to groups of the formula —OC(O)R, wherein R is an alkyl group comprising from 1 to 6 carbon atoms.

The terms “C₆₋₁₀ arylcarbonyloxy” and “C₆-C₁₀ arylcarbonyloxy,” which may be used interchangeably throughout, and as used herein, refers to a group of the formula —OC(O)R, wherein R is an aryl group comprising from 6 to 10 carbons, as defined above.

The term “heteroarylcarbonyloxy,” as used herein, refers to a group of the formula —OC(O)R, wherein R is a heteroaromatic group as defined above.

If an inventive compound or an intermediate in the present invention is a base, a desired salt may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid or ethanesulfonic acid, or the like.

If an inventive compound or an intermediate in the present inventon is an acid, a desired salt may be prepared by any suitable method known to the art, including treatment of the free acid with an inorganic or organic base, such as an amine (primary, secondary, or tertiary); an alkali metal or alkaline earth metal hydroxide; or the like. Illustrative examples of suitable salts include organic salts derived from amino acids such as glycine and arginine; ammonia; primary, secondary, and tertiary amines; and cyclic amines, such as piperidine, morpholine, and piperazine; as well as inorganic salts derived from sodium, calcium, potassium, magnesium, manganese, iron, copper, zinc, aluminum, and lithium.

The compounds of the present invention contain at least one chiral center and may exist as single stereoisomers (e.g., single enantiomers or single diastereomers), any mixture of stereoisomers (e.g., any mixture of enantiomers or diastereomers) or racemic mixtures thereof. It is specifically contemplated that, unless otherwise indicated, all stereoisomers, mixtures and racemates of the present compounds are encompassed within the scope of the present invention. Compounds identified herein as single stereoisomers are meant to describe compounds that are present in a form that contains at least from at least about 90% to at least about 99% of a single stereoisomer of each chiral center present in the compounds. Where the stereochemistry of the chiral carbons present in the chemical structures illustrated herein are not specified, it is specifically contemplated that all possible stereoisomers are encompassed therein. The compounds of the present invention may be prepared and used in stereoisomerically pure form or substantially stereoisomerically pure form. As used herein, the term “stereoisomeric” purity refers to the “enantiomeric” purity and/or “diastereomeric” purity of a compound. The term “stereoisomerically pure form,” as used herein, is meant to encompass those compounds that contain from at least about 95% to at least about 99%, and all values in between, of a single stereoisomer. The term “substantially enantiomerically pure,” as used herein is meant to encompass those compounds that contain from at least about 90% to at least about 95%, and all values in between, of a single stereoisomer. The term “diastereomerically pure,” as used herein, is meant to encompass those compounds that contain from at least about 95% to at least about 99%, and all values in between, of a single diastereoisomer. The term “substantially diastereomerically pure,” as used herein, is meant to encompass those compounds that contain from at least about 90% to at least about 95%, and all values in between, of a single diastereoisomer. The terms “racemic” or “racemic mixture,” as used herein, refer to a mixture containing equal amounts of stereoisomeric compounds of opposite configuration. For example, a racemic mixture of a compound containing one stereoisomeric center would comprise equal amount of that compound in which the stereoisomeric center is of the (S)- and (R)-configurations. The term “enantiomerically enriched,” as used herein, is meant to refer to those compositions wherein one stereoisomer of a compound is present in a greater amount than the opposite stereoisomer. Similarly, the term “diastereomerically enriched,” as used herein, refers to those compositions wherein one diastereomer of compound is present in amount greater than other diastereomer(s). The compounds of the present invention may be obtained in stereoisomerically pure (i.e., enantiomerically and/or diastereomerically pure) or substantially stereoisomerically pure (i.e., substantially enantiomerically and/or diastereomerically pure) form. Such compounds may be obtained synthetically, according to the procedures described herein using stereoisomerically pure or substantially stereoisomerically pure materials. Alternatively, these compounds may be obtained by resolution/separation of mixtures of stereoisomers, including racemic and diastereomeric mixtures, using procedures known to those of ordinary skill in the art. Exemplary methods that may be useful for the resolution/separation of stereoisomeric mixtures include derivitation with stereochemically pure reagents to form diastereomeric mixtures, chromatographic separation of diastereomeric mixtures, chromatographic separation of enantiomeric mixtures using chiral stationary phases, enzymatic resolution of covalent derivatives, and crystallization/re-crystallization. Other useful methods may be found in Enantiomers, Racemates, and Resolutions, J. Jacques et al., 1981, John Wiley and Sons, New York, N.Y., the disclosure of which is incorporated herein by reference. Preferred stereoisomers of the compounds of this invention are described herein.

If the substituents themselves are not compatible with the synthetic methods of this invention, the substituent may be protected with a suitable protecting group that is stable to the reaction conditions used in these methods. The protecting group may be removed at a suitable point in the reaction sequence of the method to provide a desired intermediate or target compound. Suitable protecting groups and the methods for protecting and de-protecting different substituents using such suitable protecting groups are well known to those skilled in the art; examples of which may be found in T. Greene and P. Wuts, Protective Groups in Organic Synthesis (3^{rd} ed.), John Wiley & Sons, New York (1999), which is incorporated herein by reference in its entirety. In some instances, a substituent may be specifically selected to be reactive under the reaction conditions used in the methods of this invention. Under these circumstances, the reaction conditions convert the selected substituent into another substituent that is either useful in an intermediate compound in the methods of this invention or is a desired substituent in a target compound.

In the compounds of this invention, R² and R^{2′}, independently or taken together, may be a suitable nitrogen protecting group. As indicated above, suitable nitrogen protecting groups are known to those of ordinary skill in the art and any nitrogen-protecting group that is useful in the methods of preparing the compounds of this invention or may be useful in the HIV protease inhibitory compounds of this invention may be used. Exemplary nitrogen protecting groups include alkyl, substituted alkyl, carbamate, urea, amide, imide, enamine, sulfenyl, sulfonyl, nitro, nitroso, oxide, phosphinyl, phosphoryl, silyl, organometallic, borinic acid and boronic acid groups. Examples of each of these groups, methods for protecting nitrogen moieties using these groups and methods for removing these groups from nitrogen moieties are disclosed in T. Greene and P. Wuts, supra. Preferably, when R² and/or R^{2′} are independently suitable nitrogen protecting groups, suitable R² and R^{2′} substituents include, but are not limited to, carbamate protecting groups such as alkyloxycarbonyl (e.g., Boc: t-butyloxycarbonyl) and aryloxycarbonyl (e.g., Cbz: benzyloxycarbonyl, or FMOC: fluorene-9-methyloxycarbonyl), alkyloxycarbonyls (e.g., methyloxycarbonyl), alkyl or arylcarbonyl, substituted alkyl, especially arylalkyl (e.g., trityl (triphenylmethyl), benzyl and substituted benzyl), and the like. When R² and R^{2′} taken together are a suitable nitrogen protecting group, suitable R²/R^{2′} substituents include phthalimido and a stabase (1,2-bis(dialkylsilyl))ethylene).

The following processes illustrate the preparation of HIV protease inhibitors according to methods of the present invention. These compounds, prepared by the methods of the present invention, are potent inhibitors of HIV protease and thus are useful in the prevention and treatment of acquired immunodeficiency syndrome (AIDS) and AIDS related complex (“ARC”).

Unless otherwise indicated, variables according to the following processes are as defined above.

Starting materials, the synthesis of which are not specifically described herein or provided with reference to published references, are either commercially available or can be prepared using methods known to those of ordinary skill in the art. Certain synthetic modifications may be done according to methods familiar to those of ordinary skill in the art.

Compounds of formula (I), wherein R¹ is phenyl substituted by at least one hydroxyl group, and Z, R², R^{2′}, R³, R⁴, R⁵, R⁶, R⁷, are as hereinbefore defined, may be prepared from compounds of formula I wherein R¹ is phenyl substituted by at least one group selected from C₁₋₆ alkylcarbonyloxy, C₆₋₁₀ arylcarbonyloxy, and heteroarylcarbonyloxy. The C₁₋₆ alkylcarbonyloxy, C₆₋₁₀ arylcarbonyloxy, and heteroarylcarbonyloxy groups may be cleaved under conditions that directly provide the desired hydroxyl substituted compounds of the invention. In general, the C₁₋₆ alkylcarbonyloxy, C₆₋₁₀ arylcarbonyloxy, and heteroarylcarbonyloxy groups may be cleaved under basic conditions, in a solvent that will not interfere with the desired transformation, and at a temperature that is compatible with the other reaction parameters, all of which are known to those of skill in the art. For example, appropriate bases include, but are not limited to, sodium bicarbonate, potassium bicarbonate, sodium carbonate, potassium carbonate, sodium hydroxide, potassium hydroxide, a sodium alkoxide such as sodium methoxide or sodium ethoxide, a potassium alkoxide such as potassium methoxide or potassium ethoxide, or a base formed in situ using an appropriate combination of reagents, such as a combination of a trialkyl or aryl amine in combination with an alkanol such as methanol. Or such a transformation may be accomplished using an acid that is known to those of skill in the art to be appropriate to cleave such a group without interfering with the desired transformation. Such acids include, but are not limited to, hydrogen halides such as hydrochloric acid or hydroiodic acid, an alkyl sulfonic acid such as methanesulfonic acid, an aryl sulfonic acid such as benzenesulfonic acid, nitric acid, sulfuric acid, perchloric acid, or chloric acid. Furthermore, appropriate solvents include those that are known to those of skill in the art to be compatible with the reaction conditions and include alkyl esters and aryl esters, alkyl, heterocyclic, and aryl ethers, hydrocarbons, alkyl and aryl alcohols, alkyl and aryl halogenated compounds, alkyl or aryl nitriles, alkyl and aryl ketones, and non-protic heterocyclic solvents. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Finally, these transformations may be conducted at temperatures from −20° C. to 100° C., depending on the specific reactants and solvents and is within the skill of one of ordinary skill in the art. Further suitable reaction conditions may be found in Greene et al., Protective Groups in Organic Synthesis; John Wiley & Sons, New York, (1999).

Compounds of formulas (I) and (X), wherein R³ is hydrogen and Z, R¹, R², R^{2′}, R⁴, R⁵, R⁶, R⁷, R⁸, and R⁹ are as hereinbefore defined, may be prepared from compounds of formulas (I) and (X) wherein R³ is a hydroxyl protecting group. The choice of a suitable hydroxy protecting group is within the knowledge of one of ordinary skill in the art. Suitable hydroxyl protecting groups that are useful in the present invention include, but are not limited to, alkyl or aryl esters, alkyl silanes, aryl silanes or alkylaryl silanes, alkyl or aryl carbonates, benzyl groups, substituted benzyl groups, ethers, or substituted ethers. The various hydroxy protecting groups can be suitably cleaved utilizing a number of reaction conditions known to those of ordinary skill in the art. The particular conditions used will depend on the particular protecting group as well as the other functional groups contained in the subject compound. Choice of suitable conditions is within the knowledge of those of ordinary skill in the art.

For example, if the hydroxy protecting group is an alkyl or aryl ester, cleavage of the protecting group may be accomplished using a suitable base, such as a carbonate, a bicarbonate, a hydroxide, an alkoxide, or a base formed in situ from an appropriate combination of agents. Furthermore, such reactions may be performed in a solvent that is compatible with the reaction conditions and will not interfere with the desired transformation. For example, suitable solvents may include alkyl esters, alkylaryl esters, aryl esters, alkyl ethers, aryl ethers, alkylaryl esters, cyclic ethers, hydrocarbons, alcohols, halogenated solvents, alkyl nitriles, aryl nitriles, alkyl ketones, aryl ketones, alkylaryl ketones, or non-protic heterocyclic compounds. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Finally, such reactions may be performed at an appropriate temperature from −20° C. to 100° C., depending on the specific reactants used. The choice of a suitable temperature is within the skill of one of ordinary skill in the art. Further suitable reaction conditions may be found in Greene et al., Protective Groups in Organic Synthesis, John Wiley & Sons, New York, (1999).

Additionally, if R³ is an alkyl silane, aryl silane or alkylaryl silane, such groups may be cleaved under conditions known to those of ordinary skill in the art. For example, such silane protecting groups may be cleaved by exposure of the subject compound to a source of fluoride ions, such as the use of an organic fluoride salt such as a tetraalkylammonium fluoride salt, or an inorganic fluoride salt. Suitable fluoride ion sources include, but are not limited to, tetramethylammonium fluoride, tetraethylammonium fluoride, tetrapropylammonium fluoride, tetrabutylammonium fluoride, sodium fluoride, and potassium fluoride. Alternatively, such silane protecting groups may be cleaved under acidic conditions using organic or mineral acids, with or without the use of a buffering agent. For example, suitable acids include, but are not limited to, hydrofluoric acid, hydrochloric acid, sulfuric acid, nitric acid, acetic acid, citric acid, and methanesulfonic acid. Such silane protecting groups may also be cleaved using appropriate Lewis acids. For example, suitable Lewis acids include, but are not limited to, dimethylbromo borane, triphenylmethyl tetrafluoroborate, and certain Pd (II) salts. Such silane protecting groups can also be cleaved under basic conditions that employ appropriate organic or inorganic basic compounds. For example, such basic compounds include, but are not limited to, sodium carbonate, potassium carbonate, sodium bicarbonate, potassium bicarbonate, sodium hydroxide, and potassium hydroxide. The cleavage of a silane protecting group may be conducted in an appropriate solvent that is compatible with the specific reaction conditions chosen and will not interfere with the desired transformation. Among such suitable solvents are, for example, alkyl esters, alkylaryl esters, aryl esters, alkyl ethers, aryl ethers, alkylaryl esters, cyclic ethers, hydrocarbons, alcohols, halogenated solvents, alkyl nitriles, aryl nitriles, alkyl ketones, aryl ketones, alkylaryl ketones, or non-protic heterocyclic compounds. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1, 4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Finally, such reactions may be performed at an appropriate temperature from −20° C. to 100° C., depending on the specific reactants used. The choice of a suitable temperature is within the skill of one of ordinary skill in the art. Further suitable reaction conditions may be found in Greene et al., Protective Groups in Organic Synthesis, John Wiley & Sons, New York, (1999).

When R³ is a benzyl or substituted benzyl ether, cleavage of the protecting group may be accomplished by treating the subject compound with hydrogen in the presence of a suitable catalyst, oxidation with suitable compounds, exposure to light of particular wavelengths, electrolysis, treatment with protic acids, or treatment with Lewis acids. The choice of particular reagents to effect such a transformation will depend on the specific subject compound used and is within the skill of one of ordinary skill in the art. For example, such benzyl or substituted benzyl ethers may be cleaved using hydrogen gas in the presence of an appropriate catalyst. Suitable catalysts include, but are not limited to, 5% palladium on carbon, 10% palladium on carbon, 5% platinum on carbon, or 10% platinum on carbon. The choice of a particular catalyst and the amounts of catalyst, the amount of hydrogen gas, and the hydrogen gas pressure used to effect the desired transformation will depend upon the specific subject compound and the particular reaction conditions utilized. Such choices are within the skill of one of ordinary skill in the art. Furthermore, such benzyl and substituted benzyl ethers may be cleaved under oxidative conditions in which a suitable amount of an oxidizer is used. Such suitable oxidizers include, but are not limited to, dichlorodicyanoquinone (DDQ), ceric ammonium nitrate (CAN), ruthenium oxide in combination with sodium periodate, iron (III) chloride, or ozone. Additionally, such ethers may be cleaved using an appropriate Lewis acid. Such suitable Lewis acids include, but are not limited to, dimethylbromo borane, triphenylmethyl tetrafluoroborate, sodium iodide in combination with trifluoroborane-etherate, trichloroborane, or tin (IV) chloride. The cleavage of a benzyl or substituted benzyl ether protecting group may be conducted in an appropriate solvent that is compatible with the specific reaction conditions chosen and will not interfere with the desired transformation. Among such suitable solvents are, for example, alkyl esters, alkylaryl esters, aryl esters, alkyl ethers, aryl ethers, alkylaryl esters, cyclic ethers, hydrocarbons, alcohols, halogenated solvents, alkyl nitriles, aryl nitriles, alkyl ketones, aryl ketones, alkylaryl ketones, or non-protic heterocyclic compounds. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Finally, such reactions may be performed at an appropriate temperature from −20° C. to 100° C., depending on the specific reactants used. The choice of a suitable temperature is within the skill of one of ordinary skill in the art. Further suitable reaction conditions may be found in Greene et al., Protective Groups in Organic Synthesis, John Wiley & Sons, New York, (1999).

When R³ is a methyl ether, cleavage of the protecting group may be accomplished by treating the subject compound with organic or inorganic acids or Lewis acids. The choice of a particular reagent will depend upon the type of methyl ether present as well as the other reaction conditions. The choice of a suitable reagent for cleaving a methyl ether is within the skill of one of ordinary skill in the art. Examples of suitable reagents include, but are not limited to, hydrochloric acid, sulfuric acid, nitric acid, para-toluenesulfonic acid, or Lewis acids such as boron trifluoride etherate. These reactions may be conducted in solvents that are compatible with the specific reaction conditions chosen and will not interfere with the desired transformation. Among such suitable solvents are, for example, alkyl esters, alkylaryl esters, aryl esters, alkyl ethers, aryl ethers, alkylaryl esters, cyclic ethers, hydrocarbons, alcohols, halogenated solvents, alkyl nitriles, aryl nitriles, alkyl ketones, aryl ketones, alkylaryl ketones, or non-protic heterocyclic compounds. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Finally, such reactions may be performed at an appropriate temperature from −20° C. to 100° C., depending on the specific reactants used. The choice of a suitable temperature is within the skill of one of ordinary skill in the art. Further suitable reaction conditions may be found in Greene et al., Protective Groups in Organic Synthesis, John Wiley & Sons, New York, (1999).

When R³ is a carbonate, cleavage of the protecting group may be accomplished by treating the subject compound with suitable basic compounds. Such suitable basic compounds may include, but are not limited to, sodium carbonate, sodium bicarbonate, potassium carbonate, potassium bicarbonate, sodium hydroxide, or potassium hydroxide. The choice of a particular reagent will depend upon the type of carbonate present as well as the other reaction conditions. These reactions may be conducted in solvents that are compatible with the specific reaction conditions chosen and will not interfere with the desired transformation. Among such suitable solvents are, for example, alkyl esters, alkylaryl esters, aryl esters, alkyl ethers, aryl ethers, alkylaryl esters, cyclic ethers, hydrocarbons, alcohols, halogenated solvents, alkyl nitriles, aryl nitriles, alkyl ketones, aryl ketones, alkylaryl ketones, or non-protic heterocyclic compounds. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Finally, such reactions may be performed at an appropriate temperature from −20° C. to 100° C., depending on the specific reactants used. The choice of a suitable temperature is within the skill of one of ordinary skill in the art. Further suitable reaction conditions may be found in Greene et al., Protective Groups in Organic Synthesis; John Wiley & Sons, New York, (1999).

Furthermore, compounds of formula (I) wherein R¹ is phenyl substituted by at least one hydroxy group, and R³ is hydrogen, may be prepared from compounds of formula (I) wherein R¹ is phenyl optionally substituted by at least one substituent independently chosen from C₁₋₄ alkylcarbonyloxy, C₁₋₁₀ arylcarbonyloxy, and heteroarylcarbonyloxy; and R³ is a hydroxyl-protecting group. In these compounds, the R¹ C₁₋₆ alkylcarbonyloxy, C₆₋₁₀ arylcarbonyloxy, and heteroarylcarbonyloxy group and the R³ hydroxyl protecting group may be removed using reactions conditions in which both groups are removed concomitantly or they may be removed in step-wise fashion. For example, when R¹ is phenyl substituted by alkylcarbonyloxy and R³ is an alkyl ester, both groups may be cleaved by reacting the subject compound with a base in an appropriate solvent and at an appropriate temperature. The choice of a suitable base, solvent, and temperature will depend on the particular subject compound and the particular protecting groups being utilized. These choices are within the skill of one of ordinary skill in the art. For example, in compound (1), wherein R¹ is phenyl substituted with methylcarbonyloxy and methyl and R³ is acetoxy, the methylcarbonyl and acetoxy protecting groups were cleaved concomitantly upon reacting compound (1) with potassium hydroxide in a mixture of methanol and acetonitrile to afford the desired compound, as shown below.

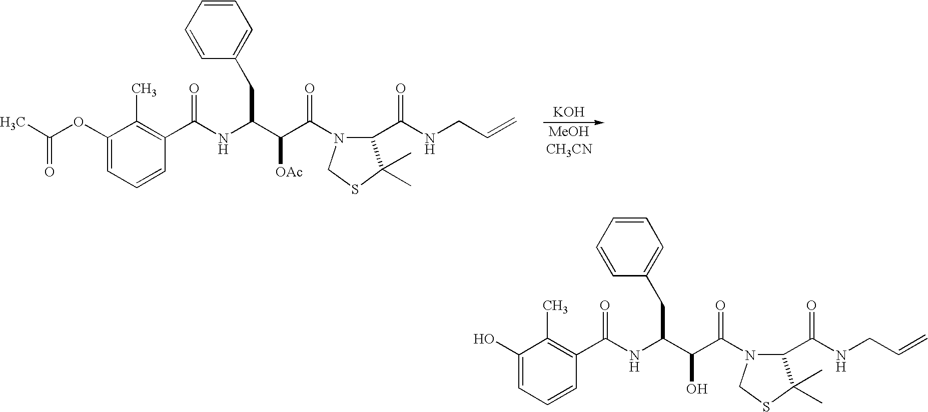

Alternatively, in compounds of formula (I) wherein R¹ is phenyl substituted by at least one group selected from C1— alkylcarbonyloxy, C₆₋₁₀ arylcarbonyloxy, and heteroarylcarbonyloxy, and R³ is a hydroxyl protecting group, the C₁₋₄ alkylcarbonyloxy, C₆₋₁₀ arylcarbonyloxy, and heteroarylcarbonyloxy group and the R³ hydroxyl protecting group may be cleaved in a stepwise manner to afford a compound of formula (I) wherein R¹ is phenyl substituted by hydroxy and R³ is hydrogen. The choice of the R³ hydroxyl protecting group and the conditions to affect its cleavage will depend upon the specific subject compound chosen and is within the knowledge of one of ordinary skill in the art. For example, in the compounds of formula (I) wherein R¹ is phenyl substituted by C₁₋₆ alkylcarbonyloxy and R³ is a silane protecting group, the R³ silane protecting group may be cleaved first by treatment of the subject compound with a fluoride source such as tetrabutylammonium fluoride in acetonitrile at room temperature, followed by cleavage of the C₁₋₆ alkylcarbonyloxy group in R¹ by treatment with a base such as potassium hydroxide in a mixture of methanol and acetonitrile at room temperature.

Compounds of formula (I) wherein Z, R¹, R², R^{2′}, R³, R⁴, R⁵, R⁶, and R⁷, are as hereinbefore defined may be prepared by reacting a compound of formula (II), wherein Y¹ is a leaving group and R¹ and R³ are as hereinbefore defined, 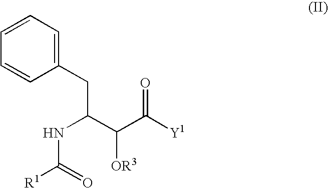
with a compound of formula (III), 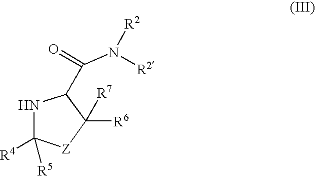
or a salt or solvate thereof.

The present invention specifically contemplates that the compounds of formula (I) may be prepared by reacting compounds of formula (III) with compounds of formula (II), wherein R³ is hydrogen, an optionally substituted C₁₋₄ alkyl group, or a suitable protecting group, such as a C₁₋₆ alkylcarbonyl, C₆₋₁₀ arylcarbonyl, or heteroarylcarbonyl group. For example, as shown below, compound (2), wherein R³ is methylcarboxy, was treated with thionyl chloride in a mixture of pyridine and acetonitrile and was then allowed to react with compound (3) to afford the desired compound (4), as shown below.

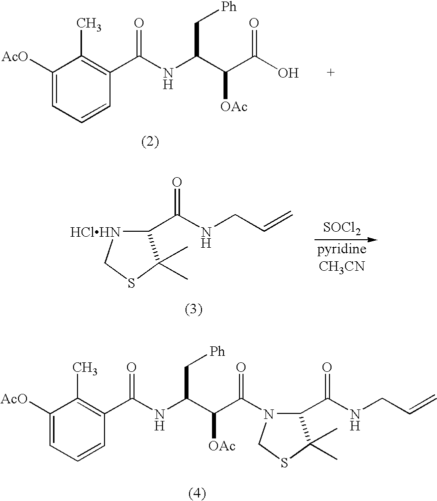

Alternatively, as shown below, compound (5), wherein R³ is hydrogen, was allowed to react with compound (3) to afford the desired product, compound (6).

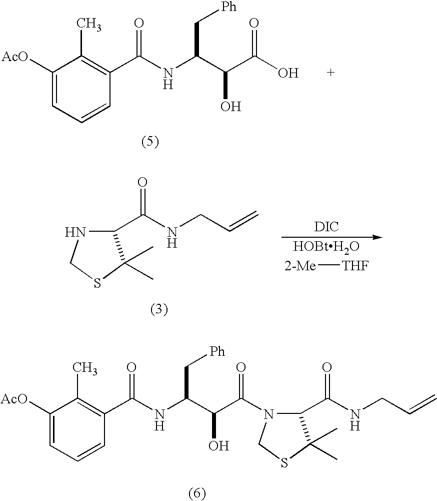

Whether R³ in the compounds of formula (II) is hydrogen, an optionally substituted C₁₋₄ alkyl group, or a suitable protecting group is dependent on the specific product compounds desired and/or the specific reaction conditions used. Such choices are within the knowledge of one of ordinary skill in the art.

For example, as shown below, compound (5) was allowed to react with acetic anhydride in ethyl acetate and methanesulfonic acid at about 70° C. to afford compound (2).

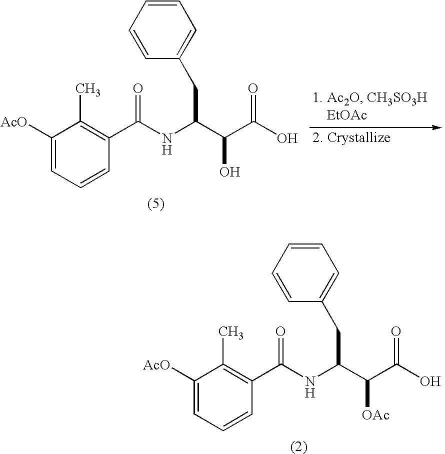

Compounds of formula (II), wherein Y¹ is hydroxy and R¹ and R³ are as hereinbefore defined, can be prepared by reaction of compounds of formula (IV), wherein Y¹ and R³ are as hereinbefore defined, with compounds of formula (V), wherein R¹ is as hereinbefore defined and Y² is hydroxy or a suitable leaving group, as shown below.

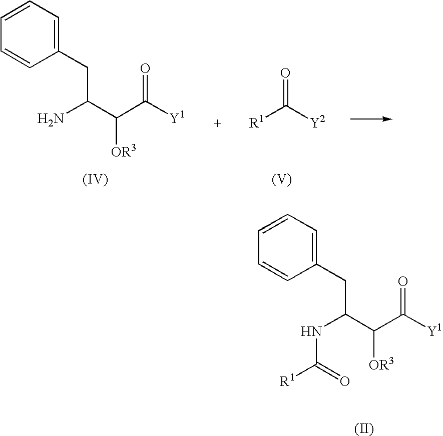

In general, these reactions may be performed in a solvent that does not interfere with the reaction, for example alkyl or aryl ethers, alkyl or aryl esters, aromatic and aliphatic hydrocarbons, non-competitive alcohols, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C., depending on the specific reactants, solvents, and other optional additives used. Such reactions may also be promoted by the addition of optional additives. Examples of such additives include, but are not limited to, hydroxybenzotriazole (HOBt), hydroxyazabenzotriazole (HOAt), N-hydroxysuccinimide (HOSu), N-hydroxy-5-norbornene-endo-2,3-dicarboximide (HONB), and 4-dimethylaminopyridine (DMAP). Whether these additives are necessary depends on the identity of the reactants, the solvent, and the temperature. Such choices are within the knowledge of one of ordinary skill in the art.

In general, the leaving group Y² in the compounds of formula (V) should be such that it provides sufficient reactivity with the amine in the compounds of formula (IV). Compounds of formula (V) that contain such suitable leaving groups may be prepared, isolated and/or purified, and subsequently reacted with the compounds of formula (IV). Alternatively, compounds of formula (V) with suitable leaving groups may be prepared and further reacted without isolation or further purification with the compounds of formula (IV) to afford compounds of formula (II). Among suitable leaving groups in the compounds of formula (V) are halides, aromatic heterocycles, sulfonic acid esters, phosphoric acid esters, anhydrides, or groups derived from the reaction of compounds of formula (V) wherein Y² is hydroxy with reagents such as carbodiimides or carbodiimide species. Examples of suitable leaving groups include, but are not limited to, chloride, iodide, imidazole, —OC(O)alkyl, —OC(O)aryl, —OC(O)Oalkyl, —OC(O)Oaryl, —OS(O₂)alkyl, —OS(O₂)aryl, —OPO(Oaryl)₂, OPO(Oalkyl)₂, and those derived from the reaction of the compounds of formula (V) wherein Y² is —OH with carbodiimides. Other suitable leaving groups are known to those of ordinary skill in the art and may be found, for example, in Humphrey, J. M.; Chamberlin, A. R. Chem. Rev., 1997, 97, 2243; Comprehensive Organic Synthesis; Trost, B. M., Ed.; Pergamon: New York, (1991); Vol. 6, pp 301-434; and Comprehensive Organic Transformations; Larock, R. C.; VCH: New York, (1989), Chapter 9.

Compounds of formula (V) where in Y² is a halogen can be prepared from compounds of formula (V) wherein Y² is hydroxy by reaction with a suitable agent. For example, the compounds of formula (V) wherein Y² is chloro may be prepared from compounds of formula (V) wherein Y² is hydroxy by reaction with agents such as thionyl chloride or oxalyl chloride. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (IV) or they may be formed in situ and reacted with the compounds of formula (IV) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art. For example, as shown below, compound (7) was allowed to react with compound (8) in a mixture of tetrahydrofuran and water, in the presence of triethylamine, at room temperature to afford the desired compound (5).

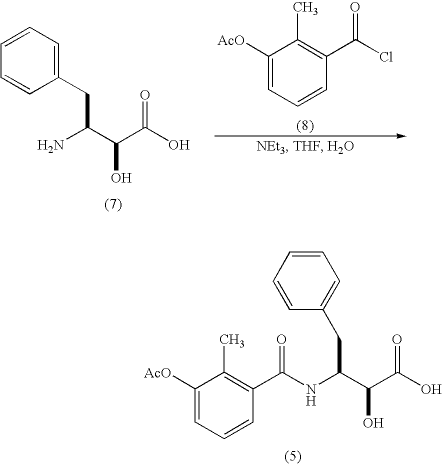

Compounds of formula (IV), wherein Y¹ is hydroxy and R³ is as defined above, are either commercially available or can be prepared by methods known to those of skill in the art.

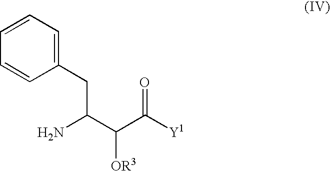

For example, the compounds of formula (IV) can be prepared as shown in the scheme below. In general, an N-protected amino acid derivative is reduced to an aldehyde using reducing agents that are suitable for such a transformation. For example, suitable reducing agents are dialkyl aluminum hydride agents, such as diisobutyl aluminum hydride for example. Another method of preparing the compounds of formula (IV) is to reduce an appropriate carboxylic acid to an alcohol with a suitable reducing agent such as LiAlH₄ or BH₃ or NaBH₄ for example, followed by oxidation of the alcohol to the corresponding aldehyde with PCC, under Swern conditions or using pyr.SO₃/DMSO/NEt₃ for example Another method of preparing the compounds of formula (IV) is to reduce an appropriate carboxylic acid derivative, such as a Weinreb amide or an acyl imidazole, using a suitable reducing agent such as LiAlH₄ or diisobutyl aluminum hydride for example. Alternatively, the compounds of formula (IV) can be prepared by the preparation of an appropriate aldehyde by reduction of the corresponding acid chloride. Next, a compound is added to the aldehyde that is the equivalent of adding a carboxylate CO₂ anion. For example, cyanide can be added to the aldehyde to afford a cyanohydrin that can then be hydrolyzed under either acidic or basic conditions to afford the desired compound, (d). Alternatively, nitromethane may be added to the aldehyde under basic conditions to afford an intermediate that is then converted into the desired compound. These compounds can be prepared according to the following procedures. In those compounds where Y³ is —CN, R. Pedrosa et al., Tetrahedron Asymm. 2001, 12, 347. For those compounds in which Y³ is CH₂NO₂, M. Shibasaki et al., Tetrahedron Lett. 1994, 35, 6123.

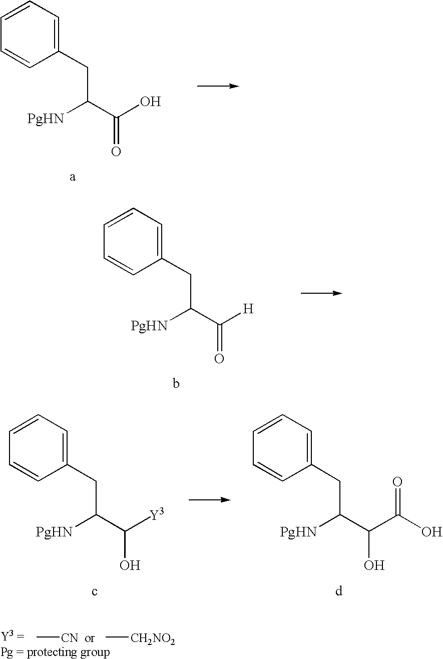

Compounds of formula (V), wherein Y² is hydroxy and R¹ is as hereinbefore defined, are either commercially available or can be prepared by methods known to those of skill in the art. For example, such compounds can be prepared from the corresponding alcohols by oxidation with suitable reagents. Such oxidation agents include, but are not limited to, KMnO₄, pyridinium dichromate (PDC), H₂Cr₂O₇ (Jones' reagent), and 2,2,6,6-tetramethylpiperidinyl-2-oxyl (TEMPO)/NaClO₂.

Compounds of formula (III), wherein Z is S, O, SO, SO₂, CH₂, or CFH, and R², R^{2′}, R⁴, R⁵, R⁶, and R⁷ are as hereinbefore defined, are either commercially available or can be prepared according to methods known to those of skill in the art. For example, see Mimoto, T. et al. J. Med. Chem. 1999, 42, 1789; EP 0751145; U.S. Pat. Nos. 5,644,028, 5,932,550, 5,962,640, 5,932,550, and 6,222,043, H. Hayashi et al., J. Med. Chem. 1999, 42, 1789; and PCT Publication No. WO 01/05230 A1, which are hereby incorporated by reference.

Alternatively, the compounds of formula (I), wherein R¹ is phenyl optionally substituted by at least one substituent independently chosen from C₁₋₆ alkyl, hydroxyl, C₁₋₆ alkylcarbonyloxy, C₆₋₁₀ arylcarbonyloxy, and heteroarylcarbonyloxy, and Z, R², R^{2′}, R¹, R⁴, R⁵, R⁶, and R⁷ are as hereinbefore defined, may be prepared by reaction of compounds of formula (VI), 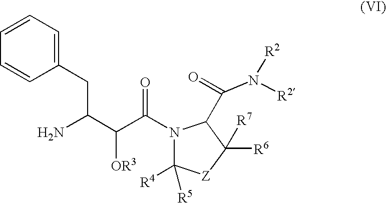
wherein Z, R², R^{2′}, R³, R⁴, R⁵, R⁶, and R⁷ are as hereinbefore defined with compounds of formula (V), wherein R¹ and Y² are as hereinbefore defined.

In general, these reactions may be performed in a solvent that does not interfere with the reaction, for example alkyl or aryl ethers, alkyl or aryl esters, aromatic and aliphatic hydrocarbons, non-competitive alcohols, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C., depending on the specific reactants, solvents, and other optional additives used. Such reactions may also be promoted by the addition of optional additives. Examples of such additives include, but are not limited to, hydroxybenzotriazole (HOBt), hydroxyazabenzotriazole (HOAt), N-hydroxysuccinimide (HOSu), N-hydroxy-5-norbornene-endo-2,3-dicarboximide (HONB), and 4-dimethylaminopyridine (DMAP). Whether these additives are necessary depends on the identity of the reactants, the solvent, and the temperature. Such choices are within the knowledge of one of ordinary skill in the art.

In general, the leaving group Y² in the compounds of formula (V) should be such that it provides sufficient reactivity with the amino group in the compounds of formula (VI). Compounds of formula (V) that contain such suitable leaving groups may be prepared, isolated and/or purified, and subsequently reacted with the compounds of formula (VI). Alternatively, compounds of formula (V) with suitable leaving groups may be prepared and further reacted without isolation or further purification with the compounds of formula (VI) to afford compounds of formula (I). Among suitable leaving groups in the compounds of formula (V) are halides, aromatic heterocycles, sulfonic acid esters, phosphoric acid esters, anhydrides, or groups derived from the reaction of compounds of formula (V) wherein Y² is hydroxy with reagents such as carbodiimides or carbodiimide species. Examples of suitable leaving groups include, but are not limited to, chloride, iodide, imidazole, —OC(O)alkyl, —OC(O)aryl, —OC(O)Oalkyl, —OC(O)Oaryl, —OS(O₂)alkyl, —OS(O₂)aryl, —OPO(Oaryl)₂, OPO(Oalkyl)₂, and those derived from the reaction of the compounds of formula (V), wherein Y² is —OH, with carbodiimides.

Compounds of formula (V) where in Y² is a halogen can be prepared from compounds of formula (V) wherein Y² is hydroxy by reaction with a suitable agent. For example, the compounds of formula (V) wherein Y² is chloro may be prepared from compounds of formula (V) wherein Y² is hydroxy by reaction with agents such as thionyl chloride or oxalyl chloride. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (VI) or they may be formed in situ and reacted with the compounds of formula (VI) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (VI), 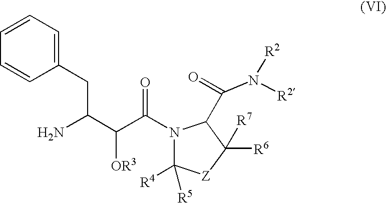
wherein Z, R², R^{2′}, R³, R⁴, R⁵, R⁶, and R⁷ are as hereinbefore defined, may be prepared from reaction of compounds of formula (VII), 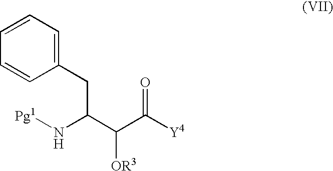
wherein Pg¹ is a suitable nitrogen protecting group, Y⁴ is hydroxy or a suitable leaving group, and R³ is as hereinbefore defined, with a compound of formula (III), wherein Z, R², R^{2′}, R⁴, R⁵, R⁶, and R⁷ are as hereinbefore defined, or a salt or solvate thereof.

A suitable protecting group Pg¹ in the compounds of formula (VII) is one that is stable to subsequent reaction conditions in which the compounds of formula (VII) are allowed to react with the compounds of formula (III). Furthermore, such a protecting group should be chosen such that it can be removed after the compounds of formula (VII) have been allowed to react with the compounds of formula (III) to afford an intermediate compound that is subsequently deprotected to afford a compound of formula (VI). Suitable protecting groups include, but are not limited to, carbamates such as t-butyloxycarbonyl and benzyloxycarbonyl, imides such as phthaloyl, or suitable benzyl groups. Such protecting groups can be introduced into the compounds of formula (VII) and subsequently removed to provide compounds of formula (VI) according to methods known to those of ordinary skill in the art and as found in, for example, Greene et al., Protective Groups in Organic Synthesis; John Wiley & Sons: New York, (1999).

In general, the leaving group Y⁴ in the compounds of formula (VII) should be such that it provides sufficient reactivity with the amino group in the compounds of formula (III). Compounds of formula (VII) that contain such suitable leaving groups may be prepared, isolated and/or purified, and subsequently reacted with the compounds of formula (III). Alternatively, compounds of formula (VII) with suitable leaving groups may be prepared and further reacted without isolation or further purification with the compounds of formula (III) to afford compounds of formula (V). Among suitable leaving groups in the compounds of formula (VII) are halides, aromatic heterocycles, sulfonic acid esters, phosphoric acid esters, anhydrides, or groups derived from the reaction of compounds of formula (VII) wherein Y⁴ is hydroxy with reagents such as carbodiimides or carbodiimide species. Examples of suitable leaving groups include, but are not limited to, chloride, iodide, imidazole, —OC(O)alkyl, —OC(O)aryl, —OC(O)Oalkyl, —OC(O)Oaryl, —OS(O₂)alkyl, —OS(O₂)aryl, —OPO(Oaryl)₂, —OPO(Oalkyl)₂, and those derived from the reaction of the compounds of formula (VII), wherein Y⁴ is —OH, with carbodiimides.

Compounds of formula (VII) where in Y⁴ is a halogen can be prepared from compounds of formula (VII) wherein Y⁴ is hydroxy by reaction with a suitable agent. For example, the compounds of formula (VII) wherein Y⁴ is chloro may be prepared from compounds of formula (VII) wherein Y⁴ is hydroxy by reaction with agents such as thionyl chloride or oxalyl chloride. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (i) or they may be formed in situ and reacted with the compounds of formula (III) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (VII) where in Y⁴ is an aromatic heterocycle can be prepared from compounds of formula (VII) wherein Y⁴ is hydroxy by reaction with a suitable agent such as carbonyl diimidazole. These compounds may be isolated and then further reacted with the compounds of formula (III) or they may be formed in situ and reacted with the compounds of formula (III) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the skill of one of ordinary skill in the art.

Compounds of formula (VII) wherein Y⁴ is —OC(O)alkyl or —OC(O)aryl may be prepared from compounds of formula (VII) wherein Y⁴ is hydroxy by reaction with suitable reagents such acyl halides, acyl imidazoles, or carboxylic acid under dehydrating conditions. Suitable reagents may include, but are not limited to, pivaloyl chloride, acetyl chloride, acetyl iodide formed in situ from acetyl chloride and sodium iodide, acetyl imidazole, or acetic acid under dehydrating conditions. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (III) or they may be formed in situ and reacted with the compounds of formula (III) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (VII) wherein Y⁴ is —OC(O)Oalkyl, —OC(O)Oaryl can be prepared from compounds of formula (VII) wherein Y⁴ is hydroxy by reaction with a suitable agents such as chloroformates of the formula Cl—C(O)Oalkyl or Cl—C(O)Oaryl. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (III) or they may be formed in situ and reacted with the compounds of formula (III) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (VII) wherein Y⁴ is —OS(O₂)alkyl or —OS(O₂)aryl can be prepared from compounds of formula (VII) wherein Y⁴ is hydroxy by reaction with a suitable agent such as an alkyl or aryl sulfonyl chloride. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (III) or they may be formed in situ and reacted with the compounds of formula (III) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Alternatively, compounds of formula (VI) may be prepared by reaction of compounds of formula (VII), wherein Y⁴ is —OH, with compounds of formula (III) under dehydrating conditions using agents such as carbodiimides or carbodiimide derived species. Such suitable agents include, but are not limited to, dicyclohexylcarbodiimide, diisopropylcarbodiimide, 1-[3-(dimethylamino)propyl]-3-ethylcarbodiimide hydrochloride (EDC), 2-chloro-4,6-dimethoxy-1,3,5-triazine (CDMT), cyanuric chloride, 4-(4,6-dimethoxy-1,3,5-triazin-2-yl)₄-methylmorpholinium chloride, O-(7-azabenzotriazol-1-yl)-N,N, N′,N′-tetramethyluronium hexafluorophosphate (HATU), carbonyldiimidazole (CDI), benzotriazole-1-yl-oxy-tris-(dimethylamino)-phosphoniumhexafluorophosphate (BOP), 2-ethoxy-1-ethoxycarbonyl-1,2-dihydroquinoline (EEDQ), 2-(1H-benzotriazole-1-yl)-1,1,3,3-tetramethyluronium hexafluorophosphate (HBTU), 2-(1H-benzotriazole-1-yl)-1,1,3,3-tetramethyluronium tetrefluoroborate (TBTU), and 3-(diethoxyphosphoryloxy)-1,2,3-benzotriazin-4(3H)-one (DEPBT). These reactions may be performed in the presence of optional additives. Suitable additives include, but are not limited to, hydroxybenzotriazole (HOBt), hydroxyazabenzotriazole (HOAt), N-hydroxysuccinimide (HOSu), N-hydroxy-5-norbornene-endo-2,3-dicarboximide (HONB), and 4-dimethylaminopyridine (DMAP). Whether these additives are necessary depends on the identity of the reactants, the solvent, and the temperature. Such choices are within the knowledge of one of ordinary skill in the art.

Alternatively, the compounds of formula (I) may be prepared by reaction of a compound of formula (VIII), 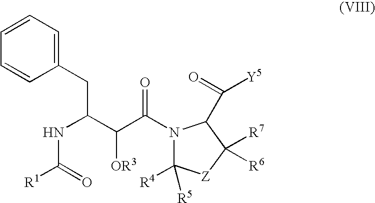
wherein Y⁵ is hydroxy or a suitable leaving group, and Z, R¹, R³, R⁴, R⁵, R⁶, and R⁷ are as hereinbefore defined, with a compound of formula (IX), 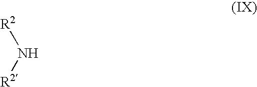
wherein R² and R^{2′} are hereinbefore defined, or a salt or solvate thereof.

In general, the leaving group Y⁵ in the compounds of formula (VIII) should be such that it provides sufficient reactivity with the amino group in the compounds of formula (IX). Compounds of formula (VIII) that contain such suitable leaving groups may be prepared, isolated and/or purified, and subsequently reacted with the compounds of formula (IX). Alternatively, compounds of formula (VIII) with suitable leaving groups may be prepared and further reacted without isolation or further purification with the compounds of formula (IX) to afford compounds of formula (I). Among suitable leaving groups in the compounds of formula (VIII) are halides, aromatic heterocycles, sulfonic acid esters, anhydrides, or groups derived from the reaction of compounds of formula (VIII) wherein Y⁵ is hydroxy with reagents such as carbodiimides or carbodiimide species. Examples of suitable leaving groups include, but are not limited to, chloride, iodide, imidazole, —OC(O)alkyl, —OC(O)aryl, —OC(O)Oalkyl, —OC(O)Oaryl, —OS(O₂)alkyl, —OS(O₂)aryl, —OPO(Oalkyl)₂, —OPO(Oaryl)₂, and those derived from the reaction of the compounds of formula (VIII), wherein Y⁵ is —OH, with carbodiimides.

Compounds of formula (VIII) where in Y⁵ is a halogen can be prepared from compounds of formula (VIII) wherein Y⁵ is hydroxy by reaction with a suitable agent. For example, the compounds of formula (VIII) wherein Y⁵ is chloro may be prepared from compounds of formula (VIII) wherein Y⁵ is hydroxy by reaction with agents such as thionyl chloride or oxalyl chloride. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (IX) or they may be formed in situ and reacted with the compounds of formula (IX) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitrites, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (VIII) where in Y⁵ is an aromatic heterocycle can be prepared from compounds of formula (VIII) wherein Y⁵ is hydroxy by reaction with a suitable agent such as carbonyl diimidazole. These compounds may be isolated and then further reacted with the compounds of formula (IX) or they may be formed in situ and reacted with the compounds of formula (IX) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (VIII) wherein Y⁵ is —OC(O)alkyl or —OC(O)aryl may be prepared from compounds of formula (VIII) wherein Y⁵ is hydroxy by reaction with suitable reagents such acyl halides, acyl imidazoles, or carboxylic acid under dehydrating conditions. Suitable reagents may include, but are not limited to, pivaloyl chloride, acetyl chloride, acetyl iodide formed in situ from acetyl chloride and sodium iodide, acetyl imidazole, or acetic acid under dehydrating conditions. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (IX) or they may be formed in situ and reacted with the compounds of formula (IX) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (VIII) wherein Y⁵ is —OC(O)Oalkyl, —OC(O)Oaryl can be prepared from compounds of formula (VIII) wherein Y⁵ is hydroxy by reaction with a suitable agents such as chloroformates of the formula Cl—C(O)Oalkyl or Cl—C(O)Oaryl. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (IX) or they may be formed in situ and reacted with the compounds of formula (IX) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (VIII) wherein Y⁵ is —OS(O₂)alkyl or —OS(O₂)aryl can be prepared from compounds of formula (VIII) wherein Y⁵ is hydroxy by reaction with a suitable agent such as an alkyl or aryl sulfonyl chloride. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (IX) or they may be formed in situ and reacted with the compounds of formula (IX) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Alternatively, compounds of formula (I) may be prepared by reaction of compounds of formula (VIII), wherein Y⁵ is —OH, with compounds of formula (IX) under dehydrating conditions using agents such as carbodiimides or carbodiimide derived species Such suitable agents include, but are not limited to, dicyclohexylcarbodiimide, diisopropylcarbodiimide, 1-[3-(dimethylamino)propyl]-3-ethylcarbodiimide hydrochloride (EDC), 2-chloro-4,6-dimethoxy-1,3,5-triazine (CDMT), cyanuric chloride, 4-(4,6-dimethoxy-1,3,5-triazin-2-yl)-4-methylmorpholinium chloride, O-(7-azabenzotriazol-1-yl)-N,N, N′,N′-tetramethyluronium hexafluorophosphate (HATU), carbonyldiimidazole (CDI), benzotriazole-1-yl-oxy-tris-(dimethylamino)-phosphoniumhexafluorophosphate (BOP), 2-ethoxy-1-ethoxycarbonyl-1,2-dihydroquinoline (EEDQ), 2-(1H-benzotriazole-1-yl)-1,1,3,3-tetramethyluronium hexafluorophosphate (HBTU), 2-(1H-benzotriazole-1-yl)-1,1,3,3-tetramethyluronium tetrefluoroborate (TBTU), and 3-(diethoxyphosphoryloxy)-1,2,3-benzotriazin-4(3H)-one (DEPBT). These reactions may be performed in the presence of optional additives. Suitable additives include, but are not limited to, hydroxybenzotriazole (HOBt), hydroxyazabenzotriazole (HOAt), N-hydroxysuccinimide (HOSu), N-hydroxy-5-norbornene-endo-2,3-dicarboximide (HONB), and 4-dimethylaminopyridine (DMAP). Whether these additives are necessary depends on the identity of the reactants, the solvent, and the temperature. Such choices are within the knowledge of one of ordinary skill in the art.

Compounds of formula (IX) are either commercially available or can be prepared by methods described herein or methods known to those of ordinary skill in the art.

The examples and preparations provided below further illustrate and exemplify the methods of the present invention. It is to be understood that the scope of the present invention is not limited in any way by the scope of the following examples. In the following examples compounds with single or multiple stereoisomeric centers, unless otherwise noted, are at least 95% stereochemically pure.

### EXAMPLES

In the examples described below, unless otherwise indicated, all temperatures in the following description are in degrees Celsius (OC) and all parts and percentages are by weight, unless indicated otherwise.

Various starting materials and other reagents were purchased from commercial suppliers, such as Aldrich Chemical Company or Lancaster Synthesis Ltd., and used without further purification, unless otherwise indicated.

The reactions set forth below were performed under a positive pressure of nitrogen, argon or with a drying tube, at ambient temperature (unless otherwise stated), in anhydrous solvents. Analytical thin-layer chromatography was performed on glass-backed silica gel 60° F. 254 plates (Analtech (0.25 mm)) and eluted with the appropriate solvent ratios (v/v). The reactions were assayed by high-pressure liquid chromotagraphy (HPLC) or thin-layer chromatography (TLC) and terminated as judged by the consumption of starting material. The TLC plates were visualized by UV, phosphomolybdic acid stain, or iodine stain.

¹H-NMR spectra were recorded on a Bruker instrument operating at 300 MHz and ¹³C-NMR spectra were recorded at 75 MHz. NMR spectra are obtained as DMSO-d₆ or CDCl₃ solutions (reported in ppm), using chloroform as the reference standard (7.25 ppm and 77.00 ppm) or DMSO-d₆ ((2.50 ppm and 39.52 ppm)). Other NMR solvents were used as needed. When peak multiplicities are reported, the following abbreviations are used: s=singlet, d=doublet, t=triplet, m=multiplet, br=broadened, dd=doublet of doublets, dt=doublet of triplets. Coupling constants, when given, are reported in Hertz.

Infrared spectra were recorded on a Perkin-Elmer FT-IR Spectrometer as neat oils, as KBr pellets, or as CDCl₃ solutions, and when reported are in wave numbers (cm⁻¹). The mass spectra were obtained using LC/MS or APCI. All melting points are uncorrected.

All final products had greater than 95% purity (by HPLC at wavelengths of 220 nm and 254 nm).

In the following examples and preparations, “Et” means ethyl, “Ac” means acetyl, “Me” means methyl, “Ph” means phenyl, (PhO)₂POCl means chlorodiphenylphosphate, “HCl” means hydrochloric acid, “EtOAc” means ethyl acetate, “Na₂CO₃” means sodium carbonate, “NaOH” means sodium hydroxide, “NaCl” means sodium chloride, “NEt₃” means triethylamine, “THF” means tetrahydrofuran, “DIC” means diisopropylcarbodiimide, “HOBt” means hydroxy benzotriazole, “H₂O” means water, “NaHCO₃” means sodium hydrogen carbonate, “K₂CO₃” means potassium carbonate, “MeOH” means methanol, “i-PrOAc” means isopropyl acetate, “MgSO₄” means magnesium sulfate, “DMSO” means dimethylsulfoxide, “AcCl” means acetyl chloride, “CH₂Cl₂” means methylene chloride, “MTBE” means methyl t-butyl ether, “DMF” means dimethyl formamide, “SOCl₂” means thionyl chloride, “H₃PO₄” means phosphoric acid, “CH₃SO₃H” means methanesulfonic acid, “Ac₂O” means acetic anhydride, “CH₃CN” means acetonitrile, and “KOH” means potassium hydroxide.

### Example 1

### Preparation of (4R)-4-allylcarbamoyl-5,5-dimethyl-thiazolidine-3-carboxylic Acid Tert-Butyl Ester

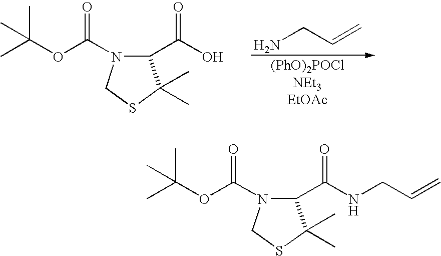 (4R)-5,5-Dimethyl-thiazolidine-3,4-dicarboxylic acid 3-tert-butyl ester (which can be prepared according to the methods of Ikunaka, M. et al., Tetrahedron Asymm. 2002, 13, 1201; Mimoto, T. et al., J. Med. Chem. 1999, 42, 1789; and Mimoto, T. et al., European Patent Application 0574135A1 (1993), 250 g; 0.957 mol) was added to an argon-purged 5-L flask and was dissolved in EtOAc (1.25 L). The solution was cooled to 2° C. and (PhO)₂POCl (208 mL; 1.00 mol) was then added in one portion. NEt₃ (280 mL; 2.01 mol) was added dropwise via addition funnel and the resulting suspension was then stirred at 0° C. Seven minutes later, allylamine (75.4 mL; 1.00 mol) was added dropwise. The ice bath was removed and the suspension was allowed to warm to room temperature. One-half hour later, 1 N HCl (750 mL; 0.750 mol) was added. The mixture was transferred to a 4-L separatory funnel using EtOAc (50 mL) for rinsing. The layers were separated. The organic fraction was washed with 7.2% aqueous Na₂CO₃ (2×1.25 L), and was then transferred to a 3-L distillation flask and was diluted with EtOAc (400 mL). The solution was dried azeotropically and concentrated to a volume of 800 mL by distillation of EtOAc at one atmosphere. After cooling to 25° C., the resulting clear yellowish EtOAc solution of (4R)-4-allylcarbamoyl-5,5dimethyl-thiazolidine-3-carboxylic acid tert-butyl ester was carried on directly into the next step. An aliquot was removed and concentrated to give (4R)-4-allylcarbamoyl-5,5-dimethyl-thiazolidine-3-carboxylic acid tert-butyl ester as a white crystalline solid: mp=94-98° C., ¹H NMR (300 MHz, CDCl₃) δ 6.12 (br s, 1H), 5.88 (app ddt, J=10.2, 17.1, 5.6 Hz, 1H), 5.28 (app dq, J=17.1, 1.5 Hz, 1H), 5.18 (app dd, J=1.2, 10.2 Hz, 1H), 4.68 (s, 2H), 4.14 (br s, 1H), 3.95 (br t, J=5.4 Hz, 2H), 1.62 (s, 3H), 1.49 (s, 9H), 1.46 (s, 3H); ¹³C NMR (75 MHz, CDCl₃) δ 170.0, 154.0, 134.4, 116.9, 82.0, 73.3, 54.0, 48.7, 42.0, 30.6, 28.6, 24.6; MS (Cl) m/z 301.1599 (301.1586 calcd for C₁₄H₂₅N₂O₃S, M+H⁺); elemental analysis calcd for C₁₄H₂₄N₂O₃S: C, 55.97; H, 8.05; N, 9.32; found: C, 56.11; H, 8.01; N, 9.11.

### Example 2

### Preparation of (4R)-5,5-dimethyl-thiazolidine-4-carboxylic Acid Allylamide

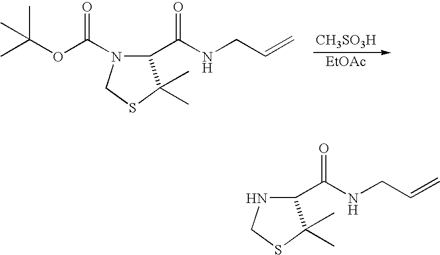

Methanesulfonic acid (155 mL; 2.39 mol) was added dropwise to the EtOAc solution of (4R)-4-allylcarbamoyl-5,5-dimethyl-thiazolidine-3-carboxylic acid tert-butyl ester in a 3-L flask. After stirring at room temperature overnight, the solution was cooled to 7° C. and H₂O (400 mL) was poured in. The mixture was transferred to a 4-L separatory funnel [using H₂O (30 mL) for rinsing] and the layers were separated. The organic fraction was extracted with H₂O (190 mL). The combined H₂O extracts were transferred to a 5-L flask and were cooled to 8° C. The pH was adjusted from 0.4 to 9.3 using 3 N NaOH (˜1.05 L). 2-Methyltetrahydrofuran (1.55 L) was poured in, followed by the addition of NaCl (150 g). The ice bath was removed and the mixture was allowed to warm to room temperature. The pH was readjusted to 9.0 using 3 N NaOH (˜1 mL). The mixture was transferred to a 4-L separatory funnel, using 2-methyltetrahydrofuran (50 mL) for rinsing, and the layers were separated. The aqueous phase was extracted with 2-methyltetrahydrofuran (950 mL). The organic extracts were vacuum-filtered through Celite directly into a 5-L distillation flask, using 2-methyltetrahydrofuran (200 mL) for rinsing. The solution was dried azeotropically and concentrated to a volume of 1.2 L by distillation of 2-methyltetrahydrofuran at one atmosphere. A measured aliquot was concentrated and weighed, which showed that 161 g of (4R)-5,5-Dimethyl-thiazolidine-4-carboxylic acid allylamide was present in solution [84% from (4R)-5,5-dimethyl-thiazolidine-3,4-dicarboxylic acid 3-tert-butyl ester]. This solution was then carried on directly into the next step. The concentrated aliquot from above yielded (4R)-5,5-Dimethyl-thiazolidine-4-carboxylic acid allylamide as a crystalline solid: mp=45-47° C., ¹H NMR (300 MHz, CDCl₃) δ 6.73 (br s, 1H), 5.87 (app ddt, J=10.2, 17.1, 5.7 Hz, 1H), 5.17-5.27 (m, 2H), 4.27 (AB q, J_{AB}=9.7 Hz, Δv=22.5 Hz, 2H), 2.94 (app tt, J=1.5, 5.8 Hz, 2H), 3.51 (s, 1H), 1.74 (s, 3H), 1.38 (s, 3H); ¹³C NMR (75 MHz, CDCl₃) δ 169.7, 134.4, 116.9, 74.8, 57.2, 51.6, 41.9, 29.1, 27.3; MS (Cl) m/z 201.1063 (201.1062 calcd for C₉H₁₇N₂OS, M+H⁺); elemental analysis calcd for C₉H₁₆N₂OS: C, 53.97; H, 8.05; N, 13.99; found: C, 53.93; H, 8.09; N, 14.07.

### Example 3

### Preparation of (2S,3S)-3-(3-acetoxy-2-methyl-benzoylamino)-2-hydroxy-4-phenyl-butyric Acid

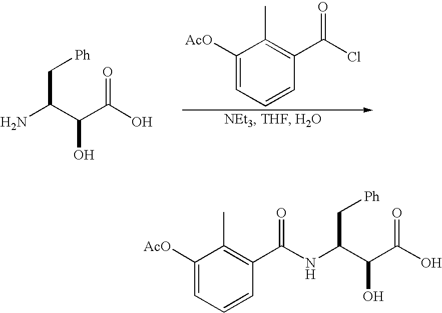

(2S,3S)-3-Amino-2-hydroxy-4-phenyl-butyric acid (which can be prepared according to the method of Pedrosa et al., Tetrahedron Asymm. 2001, 12, 347; M. Shibasaki et al., Tetrahedron Lett. 1994, 35, 6123; and Ikunaka, M. et al. Tetrahedron Asymm. 2002, 13, 1201; 185 g; 948 mmol) was added to a 5-L flask and was suspended in THF (695 mL). H₂O (695 mL) was poured in, followed by NEt₃ (277 mL; 1990 mmol). After stirring for 45 min, the solution was cooled to 6° C. A solution of acetic acid 3-chlorocarbonyl-2-methyl-phenyl ester (201 g; 948 mmol) in THF (350 mL) was then added dropwise. One-half hour later, the pH was adjusted from 8.7 to 2.5 with 6 N HCl (˜170 mL). Solid NaCl (46 g) was added, the ice bath was then removed and the mixture was stirred vigorously while warming to room temperature. The mixture was transferred to 4-L separatory funnel, using 1:1 THF/H₂O (50 mL) for the transfer, and the lower aqueous phase was then removed. The organic fraction was transferred to a 5-L distillation flask, and was then diluted with fresh THF (2.5 L). The solution was azeotropically dried and concentrated to a volume of 1.3 L by distillation of THF at one atmosphere. To complete the azeotropic drying, fresh THF (2.0 L) was added and the solution was concentrated to 1.85 L by distillation at one atmosphere and was then held at 55° C. n-Heptane (230 mL) was added dropwise via addition funnel and the solution was then immediately seeded. After crystallization had initiated, additional n-heptane (95 mL) was added dropwise. The resulting crystal slurry was stirred vigorously for 7 min. Additional n-heptane (1.52 L) was then added as a slow stream. The crystal slurry was then allowed to cool to room temperature slowly and stir overnight. The suspension was vacuum-filtered and the filter cake was then washed with 1:1 THF/n-heptane (700 mL). After drying in a vacuum oven at 45-50° C., 324 g (92%) of (2S,3S)-3-(3-acetoxy-2-methyl-benzoylamino)-2-hydroxy-4-phenyl-butyric acid was obtained as a crystalline solid contaminated with ˜7 mol % Et₃N.HCl: mp=189-191° C., ¹H NMR (300 MHz, DMSO-d₆) δ 12.65 (br s, 1H), 3.80 (d, J=9.7 Hz, 1H), 7.16-7.30 (m, 6H), 7.07 (dd, J=1.1, 8.0 Hz, 1H), 7.00 (dd, J=1.1, 7.5 Hz), 4.40-4.52 (m, 1H), 4.09 (d, J=6.0 Hz, 1H), 2.92 (app dd, J=2.9, 13.9 Hz, 1H), 2.76 (app dd, J=11.4, 13.9 Hz, 1H), 2.29 (s, 3H), 1.80 (s, 3H); ¹³C NMR (75 MHz, DMSO-d₆) δ 174.4, 169.3, 168.1, 149.5, 139.7, 139.4, 129.5, 128.3, 127.9, 126.5, 126.3, 124.8, 123.3, 73.2, 53.5, 35.4, 20.8, 12.6; MS (Cl) m/z 372.1464 (372.1447 calcd for C₂₀H₂₂NO₆, M+H⁺); elemental analysis calcd for C₂₀H₂₁NO₆.0.07 Et₃N.HCl: C, 64.34; H, 5.86; N, 3.95; Cl, 0.70; found: C, 64.27; H, 5.79; N, 3.96; Cl; 0.86.

### Example 4

### Preparation of acetic acid 3-{(1S,2S)-3-[(4R)-4-allylcarbamoyl-5,5-dimethyl-thiazolidin-3-yl]-1-benzyl-2-hydroxy-3-oxo-propylcarbamoyl}-2-methyl-phenyl Ester

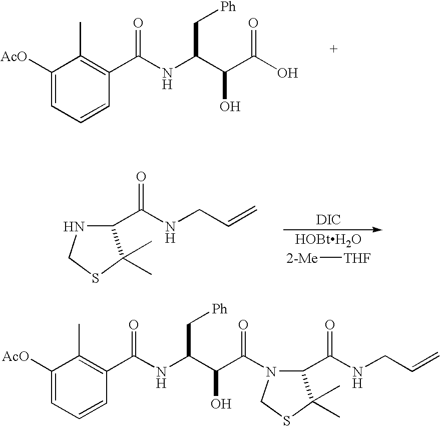

(2S,3S)-3-(3-Acetoxy-2-methyl-benzoylamino)-2-hydroxy-4-phenyl-butyric acid (271 g; 731 mmol) was added to a 5-L flask containing a solution of (4R)-5,5-Dimethyl-thiazolidine-4-carboxylic acid allylamide (161 g; 804 mmol) in 2-methyltetrahydrofuran (1.20 L total solution), while using 2-methyltetrahydrofuran (500 mL) for rinsing. HOBt.H₂O (32.6 g; 241 mmol) was added, using 2-methyltetrahydrofuran (50 mL) for rinsing. The white suspension was allowed to stir at room temperature for 10 min. Diisopropylcarbodiimide (119 mL; 760 mmol) was added in three portions (40 mL+40 mL+39 mL) at 30 min intervals. One hour after the final DIC addition, Celite (100 g) was added and the suspension was allowed to stir at room temperature for 3 h. The mixture was vacuum-filtered, while 2-methyltetrahydrofuran (400 mL) was used to rinse over the solids and wash the resulting filter cake. The filtrate was transferred to 4-L separatory funnel, using 2-methyltetrahydrofuran (50 mL) for rinsing. The solution was washed with 1 N HCl (1.25 L), and then with an aqueous solution of NaHCO₃ (27 g), NaCl (134 g) and H₂O (1.25 L). The resulting organic phase was transferred to a 3-L distillation flask and the solution was then reduced to a volume of 1.12 L by distillation of 2-methyltetrahydrofuran at one atmosphere. The solution was then diluted with 2-methyltetrahydrofuran (230 mL) to bring the total volume to 1.35 L. After cooling the solution to 23° C., the solution of crude acetic acid 3-{(1S,2S)-3-[(4R)-4-allylcarbamoyl-5,5-dimethyl-thiazolidin-3-yl]-1-benzyl-2-hydroxy-3-oxo-propylcarbamoyl}-2-methyl-phenyl ester on directly into the next step.

### Example 5

### Preparation of (4R)-3-[(2S,3S)-2-hydroxy-3-(3-hydroxy-2-methyl-benzoylamino)-4-phenyl-butyryl]-5,5-dimethyl-thiazolidine-4-carboxylic Acid Allylamide

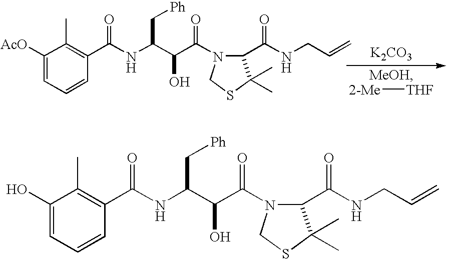

MeOH (330 mL) and K₂CO₃ (66.9 g; 484 mmol) were sequentially added to a 2-methyltetrahydrofuran solution of crude acetic acid 3-{(1S,2S)-3-[(4R)-4-allylcarbamoyl-5,5-dimethyl-thiazolidin-3-yl]-1-benzyl-2-hydroxy-3-oxo-propylcarbamoyl}-2-methyl-phenyl ester (theoretical amount: 405 g; 731 mmol) in a 3-L flask at room temperature. Two and a half hours later, additional K₂CO₃ (20 g; 144 mmol) was added. Three hours later the reaction mixture was vacuum-filtered on a pad of Celite, using 4:1 2-methyltetrahydrofuran/MeOH (330 mL) for rinsing over the solids and washing the filter cake. The filtrate was transferred to a 6-L separatory funnel, using 4:1 2-methyltetrahydrofuran/MeOH (80 mL) for rinsing. The solution was diluted with i-PrOAc (1.66 L) and was then washed with a solution of NaCl (83.0 g) in H₂O (1.60 L). The organic fraction was washed with 0.5 N HCl (1.66 L) and then with a saturated aqueous NaCl solution (400 mL). The resulting organic fraction was transferred to a 4-L Erlenmeyer flask and MgSO₄ (120 g) was added. After stirring for 10 min, the mixture was vacuum-filtered directly into a 5-L distillation flask, using 2:1 i-PrOAc/2-methyltetrahydrofuran (600 mL) for rinsing the separatory funnel and Erlenmeyer flask and washing the MgSO₄. The 2-methyltetrahydrofuran was displaced by distillation at one atmosphere with the simultaneous addition of i-PrOAc in five portions (a total of 3.60 L was used), while maintaining a minimum pot volume of ˜2.50 L. The resulting crystallizing mixture was cooled to 75° C. and was held at this temperature for 30 min. The suspension was then allowed to slowly cool to room temperature overnight. The suspension was vacuum-filtered, using i-PrOAc (600 mL) for transferring and washing the crystals. After drying in a vacuum oven at 40° C., 204 g (54% from (2S,3S)-3-(3-Acetoxy-2-methyl-benzoylamino)-2-hydroxy-4-phenyl-butyric acid) of crystalline (4R)-3-[(2S,3S)-2-Hydroxy-3-(3-hydroxy-2-methyl-benzoylamino)-4-phenyl-butyryl]-5,5-dimethyl-thiazolidine-4-carboxylic acid allylamide was obtained. This material was recrystallized as described below.

### Example 6

### Recrystallization of (4R)-3-[(2S,3S)-2-hydroxy-3-(3-hydroxy-2-methyl-benzoylamino)-4-phenyl-butyryl]-5,5-dimethyl-thiazolidine-4-carboxylic Acid Allylamide

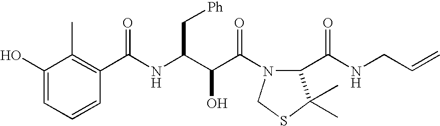

(4R)-3-[(2S,3S)-2-Hydroxy-3-(3-hydroxy-2-methyl-benzoylamino)-4-phenyl-butyryl]-5,5-dimethyl-thiazolidine-4-carboxylic acid allylamide (193 g, 378 mmol) was added to a 5-L flask and was then suspended in EtOAc (1.28 L). After heating the suspension to 76° C., MeOH (68 mL) was added and the internal temperature was then reduced to 70° C. n-Heptane (810 mL) was added dropwise to the solution, while maintaining the internal temperature at 70° C. After the n-heptane addition was complete, the resulting crystal suspension was held at 70° C. for 30 min, and was then allowed to slowly cool to room temperature overnight. The suspension was vacuum-filtered, using 1.6:1 EtOAc/n-heptane (500 mL) to transfer and wash the crystals. The crystals were then dried in a vacuum oven at 45° C. to give 162 g (84% recovery) of purified (4R)-3-[(2S,3S)-2-Hydroxy-3-(3-hydroxy-2-methyl-benzoylamino)-4-phenyl-butyryl]-5,5-dimethyl-thiazolidine-4-carboxylic acid allylamide as a white crystalline solid: mp=173-175° C., ¹H NMR (300 MHz, DMSO-d₆) displayed a ˜10:1 mixture of rotamers, major rotamer resonances δ 9.35 (s, 1H), 8.04-8.15 (m, 2H), 7.13-7.38 (m, 5H), 6.96 (t, J=7.7 Hz, 1H), 6.79 (d, J=7.2 Hz, 1H), 6.55 (d, J=7.5 Hz, 1H), 5.71-5.87 (m, 1H), 5.45 (br d, J=6.2 Hz, 1H), 4.98-5.27 (m, 4H), 4.38-4.52 (m, 3H), 3.58-3.86 (m, 2H), 2.68-2.90 (m, 2H), 1.84 (s, 3H), 1.52 (s, 3H), 1.37 (s, 3H) [characteristic minor rotamer resonances δ 9.36 (s), 8.21 (d, J=10.5 Hz), 7.82 (5, J=5.8 Hz), 4.89 (s), 4.78 (AB q, J_{AB}=9.8 Hz, Δv=27.1 Hz), 4.17-4.24 (m), 2.93-3.01 (m), 1.87 (s), 1.41 (s)]; ¹³C NMR (75 MHz, DMSO-d₆) displayed a˜10:1 mixture of rotamers, major rotamer resonances δ 170.4, 169.5, 168.2, 155.7, 139.6, 139.4, 135.5, 135.4, 129.9, 128.2, 126.2, 126.1, 121.9, 117.8, 115.6, 72.4, 72.1, 53.1, 51.4, 48.2, 41.3, 34.2, 30.5, 25.0, 12.6 [characteristic minor rotamer resonances δ 171.4, 169.7, 168.6, 139.0, 129.5, 128.4, 70.6, 54.2, 49.1, 41.5, 31.4, 24.8]; MS (Cl) m/z 512.2224 (512.2219 calcd for C₂₇H₃₄N₃O₅S, M+H⁺), elemental analysis calcd for C₂₇H₃₃N₃O₅S: C, 63.38; H, 6.50; N, 8.22; found: C, 63.19; H, 6.52; N, 8.10.

### Example 7

### Preparation of (R)-5,5-dimethyl-thiazolidine-4-carboxylic Acid Allylamide; Hydrochloride

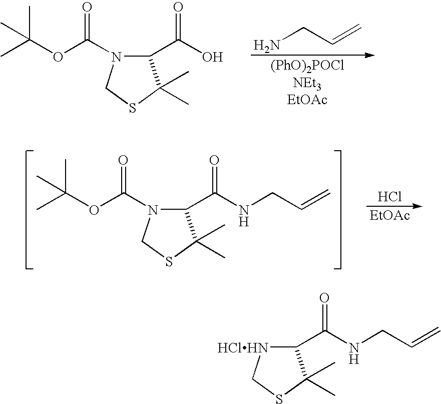

A solution of (R)-5,5-Dimethyl-thiazolidine-3,4-dicarboxylic acid 3-tert-butyl ester (105 kg, 402 mol) and ethyl acetate (690 L) was treated with diphenylchlorophosphate (113 kg, 422 mol) and was then cooled to 0° C. NEt₃ (85.5 kg, 844 mol) was added while maintaining the temperature at 5° C., and the mixture was then held at this temperature for 2 h. The mixture was cooled to 0° C., and allylamine (24.1 kg, 422 mol) was then added while maintaining the temperature at 5° C. The mixture was warmed to 20° C. and was then quenched with 10 wt. % aqueous HCl (310 L). After separation of the layers, the organic fraction was washed with 8.6 wt. % aqueous Na₂CO₃ (710 L). After separation of the layers, the aqueous fraction was extracted with ethyl acetate (315 L). The combined ethyl acetate extracts containing AG-074278 were dried by azeotropic distillation at one atmosphere, while maintaining a minimum pot volume of approximately 315 L. The resulting suspension of (R)-4-Allylcarbamoyl-5,5-dimethyl-thiazolidine-3-carboxylic acid tert-butyl ester was cooled to 5° C. A 13 wt. % solution of anhydrous HCl (36.8 kg, 1008 mol) in ethyl acetate (263 L) was cooled to 5° C. and was then added to the (R)-4-Allylcarbamoyl-5,5-dimethyl-thiazolidine-3-carboxylic acid tert-butyl ester suspension while maintaining the temperature at 15° C. The resulting suspension was held at 20° C. for 19 h, and was then cooled and held at 5° C. for 2 h. The suspension was then filtered, using cold ethyl acetate for rinsing. The wet cake was dried under vacuum at 45° C. to give 90.5 kg (95.2%) of (R)-5,5-Dimethyl-thiazolidine-4-carboxylic acid allylamide hydrochloride as a white solid: ¹H NMR (300 MHz, DMSO-d₆) δ 8.94 (app t, J=5.5 Hz, 1H), 5.82 (ddt, J=10.4, 17.2, 5.2 Hz, 1H), 5.19-5.25 (m, 1H), 5.10-5.14 (m, 1H), 4.38 (AB q, J_{AB}=9.8 Hz, Δv=14.5 Hz, 2H), 4.08 (s, 1H), 3.72-3.91 (m, 2H), 1.58 (s, 3H), 1.32 (s, 3H); ¹³C NMR (75 MHz, DMSO-d₆) δ 161.7, 132.2, 114.0, 67.9, 51.4, 43.5, 39.3, 25.3, 24.3; MS (Cl) m/z 201.1070 (201.1062 calcd for C₉H₁₇N₂OS, M+H⁺); elemental analysis calcd for C₉H₁₇ClN₂OS: C, 45.65; H, 7.24; N, 11.83; Cl, 14.97; found: C, 45.41; H, 7.33; N, 11.69; Cl, 15.22.

### Example 8

### Preparation of (2S,3S)-2-acetoxy-3-(3-acetoxy-2-methyl-benzoylamino)₄-phenyl-butyric Acid

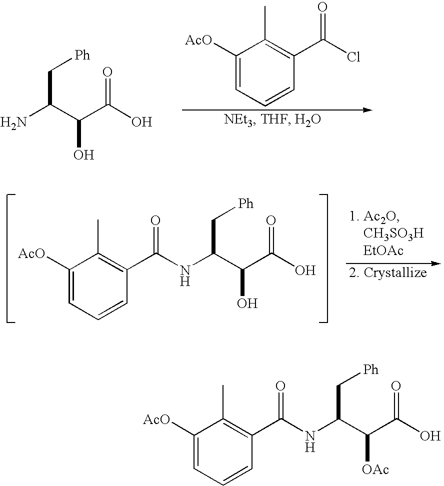

A mixture of (2S,3S)-3-Amino-2-hydroxy-4-phenyl-butyric acid (110 kg, 563 mol), NaCl (195 kg), and THF (413 L) was charged with NEt₃ (120 kg, 1183 mol) and H₂O (414 L) at ambient temperature. The resulting mixture was cooled to 0° C. Acetic acid 3-chlorocarbonyl-2-methyl-phenyl ester (120 kg, 563 mol) was added to a separate reactor and was then dissolved in THF (185 L). The resulting solution of acetic acid 3-chlorocarbonyl-2-methyl-phenyl ester was cooled to 10° C., and was then added to the (2S,3S)-3-amino-2-hydroxy-4-phenyl-butyric acid mixture while maintaining the temperature <10° C. during addition. The resulting biphasic mixture was agitated at 5° C. for 1 h, and was then adjusted to pH 2.5-3.0 with concentrated HCl (62 kg). The mixture was then warmed to 25° C., and the layers were separated. The resulting THF fraction, containing (2S,3S)-3-(3-acetoxy-2-methyl-benzoylamino)-2-hydroxy-4-phenyl-butyric acid, was partially concentrated by distillation at one atmosphere. THF was then replaced with ethyl acetate by distillation at one atmosphere, while maintaining a minimum pot volume of 1500 L. The resulting solution was cooled to 25° C., and was then charged with acetic anhydride (74.8 kg, 733 mol) and methanesulfonic acid (10.8 kg, 112 mol). The mixture was heated at 70° C. for approximately 3 h. The mixture was cooled to 25° C., and was then quenched with H₂O (1320 L) while maintaining the temperature at 20° C. After removal of the aqueous layer, the organic fraction was charged with ethyl acetate (658 L) and H₂O (563 L). After agitation, the aqueous phase was removed. The organic fraction was washed twice with 13 wt. % aqueous NaCl (2×650 L). The organic fraction was partially concentrated and dried by vacuum distillation (70-140 mm Hg) to a volume of approximately 1500 L. The resulting solution was heated to 40° C., and was then charged with n-heptane (1042 L) while maintaining the temperature at 40° C. The solution was seeded with (2S,3S)-2-acetoxy-3-(3-acetoxy-2-methyl-benzoylamino)₄-phenyl-butyric acid (0.1 kg), and additional n-heptane (437 L) was then added slowly. The crystallizing mixture was maintained at 40° C. for 1 h. Additional n-heptane (175 L) was added while maintaining the temperature at 40° C. The crystalline suspension was cooled and held at 25° C. for 1 h, then at 0° C. for 2 h. The suspension was filtered, using n-heptane for rinsing. The wet cake was dried under vacuum at 55° C. to give 174 kg (74.5%) of (2S,3S)-2-acetoxy-3-(3-acetoxy-2-methyl-benzoylamino)-4-phenyl-butyric acid as, a white solid: m.p.=152-154° C.; ¹H NMR (300 MHz, CDCl₃) δ 7.21-7.35 (m, 5H), 7.13 (app t, J=7.9 Hz, 1H), 7.01 (app d, J=8.1 Hz, 1H), 6.94 (app d, J=7.2 Hz, 1H), 5.99 (d, J=9.0 Hz, 1H), 5.33 (d, J=4.1 Hz, 1H), 4.96-5.07 (m, 1H), 3.07 (dd, J=5.5, 14.6 Hz, 1H), 2.90 (dd, J=10.0, 14.5 Hz, 1H), 2.30 (s, 3H), 2.18 (s, 3H), 1.96 (s, 3H); ¹³C NMR (125 MHz, CDCl₃) δ 170.4, 170.2, 169.6, 169.5, 149.5, 137.81, 136.5, 129.2, 128.6, 128.4, 127.0, 126.6, 124.5, 123.7, 73.1, 50.9, 35.9, 20.6, 20.5, 12.4; elemental analysis calcd for C₂₂H₂₃NO₇: C, 63.92; H, 5.61; N, 3.39; found: C, 64.22; H, 5.68; N, 3.33; MS (Cl) m/z 414.1572 (414.1553 calcd for C₂₂H₂₄NO₇, M+H⁺).

### Example 9

### Preparation of (4R)-3-[(2S,3S)-2-hydroxy-3-(3-hydroxy-2-methyl-benzoylamino)-4-phenyl-butyryl]-5,5-dimethyl-thiazolidine-4-carboxylic Acid Allylamide

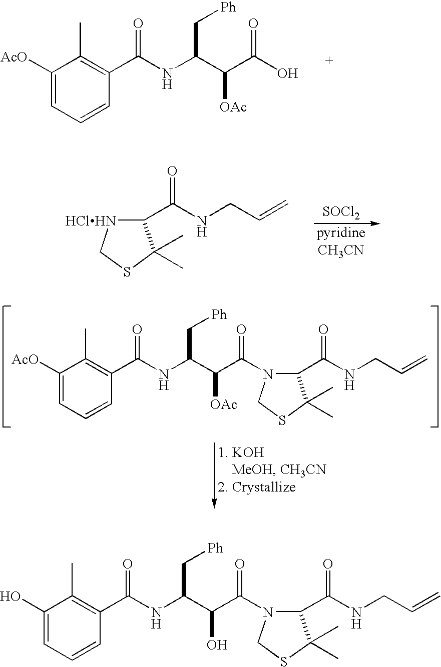

A solution of (2S,3S)-2-acetoxy-3-(3-acetoxy-2-methyl-benzoylamino)-4-phenyl-butyric acid (140 kg, 339 mol), CH₃CN (560 L), and pyridine (64.3 kg, 813 mol) was cooled to 15° C. SOCl₂ (44.3 kg, 373 mol) was charged while maintaining the temperature at 15° C. The mixture was held at 15° C. for 1 h. A separate reactor was charged with (R)-5,5-dimethyl-thiazolidine-4-carboxylic acid allylamide hydrochloride (96.6 kg, 408 mol), CH₃CN (254 L), and pyridine (29.5 kg, 373 mol), and was then cooled to 15° C. The (2S,3S)-2-acetoxy-3-(3-acetoxy-2-methyl-benzoylamino)-4-phenyl-butyric acid chloride solution was added to the (R)-5,5-dimethyl-thiazolidine-4-carboxylic acid allylamide solution, while maintaining the temperature at 15° C. The mixture was held at 15° C. for 6 h. A separate reactor was charged with KOH (167 kg, 2709 mol) and methanol (280 L) using a 0° C. cooling jacket. The resulting KOH/methanol solution was cooled to 5° C. The crude acetic acid 3-{(1S,2S)-2-acetoxy-3-[(R)-4-allylcarbamoyl-5,5-dimethyl-thiazolidin-3-yl]-1-benzyl-3-oxo-propylcarbamoyl}-2-methyl-phenyl ester mixture was added to the KOH/methanol solution while maintaining the temperature at 10° C. After addition was complete, the mixture was held at 25° C. for 3 h. The mixture was charged with H₂O (840 L) and ethyl acetate (840 L), and was then followed by acidification to pH 5-6.5 with concentrated HCl (85 kg) while maintaining the temperature at 20° C. The resulting layers were separated. The organic fraction was sequentially washed with 6.8 wt. % aqueous NaHCO₃ (770 L), an aqueous HCl/NaCl solution (H₂O: 875 L; conc. HCl: 207 kg; NaCl: 56 kg), 8.5 wt. % aqueous NaHCO₃ (322 L), and then with 3.8 wt. % aqueous NaCl (728 L). The resulting organic fraction was partially concentrated by distillation at one atmosphere. The solvent was exchanged with ethyl acetate by continuing distillation and maintaining the pot temperature at ≧70° C. Ethyl acetate was added such that the pot volume remained at approximately 840 L. The solution was then cooled to 20° C. and held at this temperature until crystallization was observed. n-Heptane (280 L) was added and the suspension was agitated at 15° C. for 4 h. The crystals were, using cold 2.4:1 (v/v) ethyl acetate/n-heptane for rinsing. The wet cake was dried under vacuum at 45° C. to provide crude (R)-3-[(2S,3S)-2-hydroxy-3-(3-hydroxy-2-methyl-benzoylamino)-4-phenyl-butyryl]-5,5-dimethyl-thiazolidine-4-carboxylic acid allylamide. Decolorization and recrystallization was conducted as follows: A mixture of crude (R)-3-[(2S,3S)-2-hydroxy-3-(3-hydroxy-2-methyl-benzoylamino)-4-phenyl-butyryl]-5,5-dimethyl-thiazolidine-4-carboxylic acid allylamide, ADP carbon (21 kg), Supercel (3 kg), and ethyl acetate (780 L) was heated to 70° C. CH₃OH (40 L) was added to the mixture. The mixture was filtered, and the resulting clear filtrate was heated to reflux at one atmosphere to begin distillation. CH₃OH was displaced as follows: ethyl acetate (388 L) was charged while maintaining the pot volume at approximately 840 L and at 70° C. The solution was slowly charged with n-heptane (316 L), while maintaining a temperature of 70° C. The mixture was then cooled to 20° C. and was held at this temperature for 4 h. The crystals were filtered, using cold 2.1:1 (v/v) ethyl acetate/n-heptane for rinsing. The wet cake was dried under vacuum at 45° C. to give 103 kg (59.6%) of (4R)-3-[(2S,3S)-2-hydroxy-3-(3-hydroxy-2-methyl-benzoylamino)-4-phenyl-butyryl]-5,5-dimethyl-thiazolidine-4-carboxylic acid allylamide as a white crystalline solid: mp=173-175° C., ¹H NMR (300 MHz, DMSO-d₆) displayed a˜10:1 mixture of rotamers, major rotamer resonances δ 9.35 (s, 1H), 8.04-8.15 (m, 2H), 7.13-7.38 (m, 5H), 6.96 (t, J=7.7 Hz, 1H), 6.79 (d, J=7.2 Hz, 1H), 6.55 (d, J=7.5 Hz, 1H), 5.71-5.87 (m, 1H), 5.45 (br d, J=6.2 Hz, 1H), 4.98-5.27 (m, 4H), 4.38-4.52 (m, 3H), 3.58-3.86 (m, 2H), 2.68-2.90 (m, 2H), 1.84 (s, 3H), 1.52 (s, 3H), 1.37 (s, 3H) [characteristic minor rotamer resonances δ 9.36 (s), 8.21 (d, J=10.5 Hz), 7.82 (5, J=5.8 Hz), 4.89 (s), 4.78 (AB q, J_{AB}=9.8 Hz, Δv=27.1 Hz), 4.17-4.24 (m), 2.93-3.01 (m), 1.87 (s), 1.41 (s)]; ¹³C NMR (75 MHz, DMSO-d₆) displayed a ˜10:1 mixture of rotamers, major rotamer resonances δ 170.4, 169.5, 168.2, 155.7, 139.6, 139.4, 135.5, 135.4, 129.9, 128.2, 126.2, 126.1, 121.9, 117.8, 115.6, 72.4, 72.1, 53.1, 51.4, 48.2, 41.3, 34.2, 30.5, 25.0, 12.6 [characteristic minor rotamer resonances δ 171.4, 169.7, 168.6, 139.0, 129.5, 128.4, 70.6, 54.2, 49.1, 41.5, 31.4, 24.8]; MS (Cl) m/z 512.2224 (512.2219 calcd for C₂₇H₃₄N₃O₅S, M+H⁺), elemental analysis calcd for C₂₇H₃₃N₃O₅S: C, 63.38; H, 6.50; N, 8.22; found: C, 63.19; H, 6.52; N, 8.10.

### Example 10

### Preparation of (2S,3S)-3-Amino-2-hydroxy-4-phenyl-butyric Acid; Hydrochloride

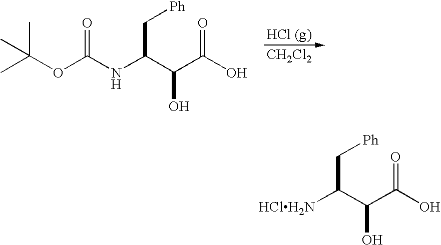

HCl gas (51 g, 1.4 mol) was bubbled into a suspension of (2S,3S)-3-tert-butoxycarbonylamino-2-hydroxy-4-phenyl-butyric acid (163 g, 551 mmol) and CH₂Cl₂ (2.0 L) at 0° C. The resulting off-white suspension was allowed to warm to ambient temperature and stir overnight. ¹H NMR analysis of a concentrated aliquot showed approximately 95% conversion to product. The suspension was cooled to 0° C., and additional HCl gas (46 g, 1.3 mol) was bubbled into the suspension. After warming to ambient temperature, the suspension was stirred overnight. The suspension was vacuum-filtered, the solid was rinsed with CH₂Cl₂ (200 mL), and the solid was then dried in a vacuum oven at 45° C. for 24 h to give 129 g (100%) of (2S,3S)-3-amino-2-hydroxy-4-phenyl-butyric acid; hydrochloride as a white solid: ¹H NMR (300 MHz, DMSO-d₆) δ 13.05 (br s, 1H), 8.25 (br s, 3H), 7.22-7.34 (m, 5H), 4.41 (d, J=2.6 Hz, 1H), 3.66 (br s, 1H), 2.84 (AB portion of ABX, J_{AX}=11.0 Hz, J_{BX}=2.8 Hz, Δv=19.6 Hz, 2H); ¹³C NMR (75 MHz, DMSO-d₆) δ 172.4, 136.6, 129.8, 128.7, 127.1, 69.6, 55.0, 33.6; MS (Cl) m/z 196.0979 (196.0974 calcd for C₁₀H₁₄NO₃, M-Cl^{-).}

### Example 11

### Preparation of (2S,3S)-3-(3-Acetoxy-2-methyl-benzoylamino)-2-hydroxy-4-phenyl-butyric Acid

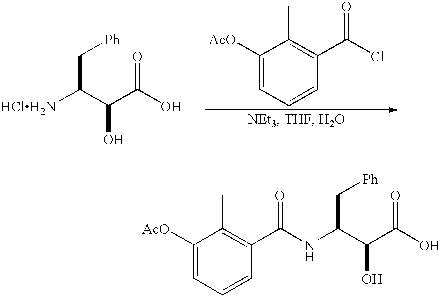

NEt₃ (186 mL, 1.34 mol) was added to a suspension of (2S,3S)-3-amino-2-hydroxy-4-phenyl-butyric acid; hydrochloride (100 g, 432 mmol), H₂O (320 mL), and tetrahydrofuran (320 mL). The suspension was cooled to 4° C. and a solution of acetic acid 3-chlorocarbonyl-2-methyl-phenyl ester (93.6 g, 440 mmol) and THF (160 mL) was added dropwise. The resulting solution was warmed to ambient temperature and stir for 1 h. The solution was cooled to 10° C. and the pH was adjusted to 2.0 using 6 N HCl (87 mL). NaCl (25 g) and tetrahydrofuran (200 mL) were added, and the mixture was warmed to ambient temperature. The phases were separated and the tetrahydrofuran fraction was dried over MgSO₄ and filtered. The filtrate was concentrated to a volume of 330 mL using a rotary evaporator, and was then diluted with tetrahydrofuran (230 mL). n-Heptane (1.2 L) was added slowly and the resulting white suspension of solid was stirred at ambient temperature overnight. The suspension was vacuum-filtered, the solid was rinsed with n-heptane (2×500 mL), and the solid was dried in a vacuum oven at 45° C. for 24 h to give 150 g (93.6%) of (2S,3S)-3-(3-acetoxy-2-methyl-benzoylamino)-2-hydroxy-4-phenyl-butyric acid as a white solid that was contaminated with ˜7.7 mol % Et₃N.HCl: mp=189-191° C., ¹H NMR (300 MHz, DMSO-d₆) δ 12.65 (br s, 1H), 3.80 (d, J=9.7 Hz, 1H), 7.16-7.30 (m, 6H), 7.07 (dd, J=1.1, 8.0 Hz, 1H), 7.00 (dd, J=1.1, 7.5 Hz), 4.40-4.52 (m, 1H), 4.09 (d, J=6.0 Hz, 1H), 2.92 (app dd, J=2.9, 13.9 Hz, 1H), 2.76 (app dd, J=11.4, 13.9 Hz, 1H), 2.29 (s, 3H), 1.80 (s, 3H); ¹³C NMR (75 MHz, DMSO-d₆) δ 174.4, 169.3, 168.1, 149.5, 139.7, 139.4, 129.5, 128.3, 127.9, 126.5, 126.3, 124.8, 123.3, 73.2, 53.5, 35.4, 20.8, 12.6; MS (Cl) m/z 372.1464 (372.1447 calcd for C₂₀H₂₂NO₆, M+H⁺).

### Example 12

### Preparation of (4R)-3-[(2S,3S)-2-Hydroxy-3-(3-hydroxy-2-methyl-benzoylamino)-4-phenyl-butyryl]-5,5-dimethyl-thiazolidine-4-carboxylic acid 2-methyl-benzylamide

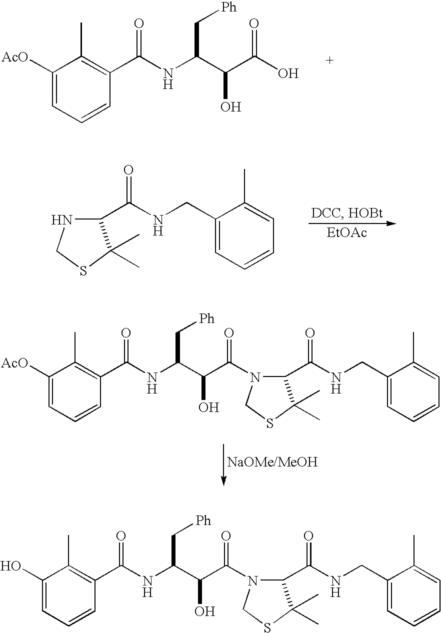

A solution of dicyclohexylcarbodiimide (3.05 g, 14.8 mmol) was added dropwise to a suspension of (2S,3S)-3-(3-acetoxy-2-methyl-benzoylamino)-2-hydroxy-4-phenyl-butyric acid (5.00 g, 13.5 mmol), (4R)-5,5-dimethyl-thiazolidine-4-carboxylic acid 2-methyl-benzylamide (3.74 g, 14.1 mmol), HOBt.H₂O (1.82 g, 13.5 mmol), and ethyl acetate (100 mL) at ambient temperature. After stirring at ambient temperature overnight, the suspension was vacuum filtered. The filtrate was sequentially washed with 5% aqueous Na₂CO₃ (50 mL), 1 N HCl (50 mL), and half-saturated aqueous NaCl (50 mL). After drying over Na₂SO₄, the ethyl acetate solution of acetic acid 3-{(1S,2S)-1-benzyl-3-[(4R)-5,5-dimethyl-4-(2-methyl-benzylcarbamoyl)-thiazolidin-3-yl]-2-hydroxy-3-oxo-propylcarbamoyl}-2-methyl-phenyl ester was concentrated to a volume of approximately 15 mL using a rotary evaporator. Methanol (11 mL) was added, and the solution was then cooled to 0° C. NaOMe (3.1 mL of a 25 wt. % solution in methanol, 13.5 mmol) was added dropwise, and the resulting mixture was stirred at 0° C. for 1 h. Ethyl acetate (108 mL) was added, and 0.15 N HCl (108 mL) was then added slowly. The mixture was warmed to ambient temperature and the layers were separated. The organic fraction was washed with 2.5% aqueous Na₂CO₃ (30 mL) and then with a solution of NaCl (6.6 g) and 0.1 N HCl (30 mL). The resulting organic fraction was dried over Na₂SO₄, filtered, and then concentrated to a volume of approximately 21 mL using a rotary evaporator. Ethyl acetate (15 mL) was added, followed by the slow addition of n-heptane (75 mL). The resulting suspension was stirred overnight, and was then vacuum-filtered. The solid was rinsed with n-heptane (2×25 mL), and was then dried in a vacuum oven at 45° C. for 24 h to give 7.41 g (95.4%) of (4R)-3-[(2S,3S)-2-hydroxy-3-(3-hydroxy-2-methyl-benzoylamino)-4-phenyl-butyryl]-5,5-dimethyl-thiazolidine-4-carboxylic acid 2-methyl-benzylamide: ¹H NMR (300 MHz, DMSO-d₆) displayed a ˜7:1 mixture of rotamers, major rotamer resonances δ 9.37 (s, 1H), 8.32 (t, J=5.6 Hz, 1H), 8.14 (d, J=8.3 Hz, 1H), 7.10-7.34 (m, 9H), 6.95 (t, J=7.7 Hz, 1H), 6.78 (d, J=7.7 Hz, 1H), 6.56 (d, J=7.1 Hz, 1H), 5.46 (br s, 1H), 5.08 (ABq, J_{AB}=9.1 Hz, 2H), 4.38-4.50 (m, 3H), 4.11 (dd, J=4.7, 15.1 Hz, 1H), 2.85 (app dd, J=2.8, 13.6 Hz, 1H), 2.73 (app dd, J=10.5, 13.5 Hz, 1H), 2.27 (s, 3H), 1.84 (s, 3H), 1.50 (s, 3H), 1.36 (s, 3H) [characteristic minor rotamer resonances δ 8.19 (d, J=8.5 Hz), 8.07 (t, J=5.7 Hz), 6.49 (d, J=7.5 Hz), 4.93 (s), 4.80 (ABq, J_{AB}=9.7 Hz), 1.82 (s), 1.40 (s)]; ¹³C NMR (75 MHz, DMSO-d₆) displayed a ˜7:1 mixture of rotamers, major rotamer resonances δ 170.5, 169.5, 168.3, 155.7, 139.7, 139.4, 137.1, 136.0, 130.2, 129.9, 128.3, 128.2, 127.2, 126.2, 126.1, 126.0, 121.8, 117.8, 115.6, 72.4, 71.9, 53.2, 51.5, 48.1, 40.8, 34.2, 30.6, 25.0, 19.1, 12.6 [characteristic minor rotamer resonances δ 171.4, 169.6, 168.8, 139.0, 137.0, 135.8, 129.5, 128.4, 71.9, 54.3, 31.5, 24.8]; MS (Cl) m/z 576.2552 (576.2532 calcd for C₃₂H₃₈N₃O₅S, M+H⁺).

### Example 13

### Preparation of (2S,3S)-3-Amino-2-hydroxy-4-phenyl-butyric Acid Ethyl Ester; Hydrochloride

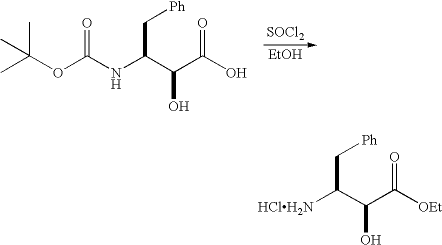

SOCl₂ (49.4 mL, 677 mmol) was added dropwise to absolute ethanol (500 mL), which had been cooled to 2° C. with an ice bath. After stirring the resulting solution for 0.5 h, (2S,3S)-3-tert-butoxycarbonylamino-2-hydroxy-4-phenyl-butyric acid (50.0 g, 169 mmol) was added as a solid. After stirring the resulting suspension for 20 min, the ice bath was removed and the mixture was warmed to ambient temperature. Two hours later, the flask was submerged in an oil bath and the yellowish solution was heated at reflux overnight. The flask was then equipped with a distillation head, the oil bath temperature was increased to 85-90° C., and 375 mL of distillate (ethanol) was collected (b.p.=76-82° C., 1 atm) and was discarded. The yellowish solution remaining in the boiling pot was allowed to cool to 35° C. Methyl t-butyl ether (400 mL) was slowly added, followed by the addition of n-heptane (100 mL). The resulting suspension was allowed to stir overnight and was then vacuum-filtered. The solid was rinsed with n-heptane (3×150 mL) and was then dried in a vacuum oven at 45° C. overnight to give 40.2 g (91.3%) of (2S,3S)-3-amino-2-hydroxy-4-phenyl-butyric acid ethyl ester; hydrochloride as an off-white solid: mp=129.5-131.5° C.; ¹H NMR (300 MHz, DMSO-d₆) δ 8.46 (br s, 3H), 7.19-7.33 (m, 5H), 6.37 (d, J=5.2 Hz, 1H), 4.48 (dd, J=2.4, 5.0 Hz, 1H), 3.68-3.82 (m, 2H), 3.56 (app dq, J=10.7, 7.1 Hz, 1H), 2.82-2.95 (m, 2H), 1.00 (t, J=7.1 Hz, 3H); ¹H NMR (300 MHz, D₂O) δ 7.19-7.33 (m, 5H), 4.51 (d, J=2.8 Hz, 1H), 4.06 (dt, J=2.8, 7.5 Hz, 1H), 3.85 (app dq, J=10.6, 7.2 Hz, 1H), 3.68 (app dq, J=10.6, 7.2 Hz, 1H), 2.83-2.97 (m, 2H), 1.07 (t, J=7.1 Hz, 3H); ¹³C NMR (75 MHz, DMSO-d₆) δ 170.7, 136.5, 129.9, 128.5, 127.1, 69.0, 60.8, 54.8, 33.3, 14.0; MS (Cl) m/z 224.1297 (224.1287calcd for C₁₂H₁₈NO₃, M-Cl^{-).}

### Example 14

### Preparation of (2S,3S)-3-(3-Acetoxy-2-methyl-benzoylamino)-2-hydroxy-4-phenyl-butyric Acid Ethyl Ester

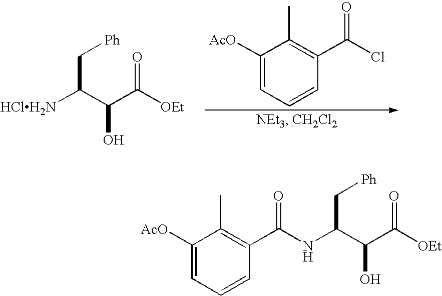

NEt₃ (63.0 mL, 450 mmol) was added to an ambient temperature suspension of (2S,3S)-3-amino-2-hydroxy-4-phenyl-butyric acid ethyl ester; hydrochloride (38.9 g, 150 mmol) and CH₂Cl₂ (800 mL), and the resulting solution was cooled to 1° C. A solution of acetic acid 3-chlorocarbonyl-2-methyl-phenyl ester (35.0 g, 165 mmol) and CH₂Cl₂ (150 mL) was slowly added, and the resulting white suspension was then allowed to warm to ambient temperature and stir overnight. 0.5 N HCl (400 mL) was added and the resulting layers were separated. The organic fraction was washed with H₂O (400 mL), and then with one-quarter saturated aqueous NaHCO₃. Methanol (40 mL) was added to the organic fraction, which was then dried over MgSO₄ and filtered. The filtrate was concentrated to a solid with a rotary evaporator, and was further dried in a vacuum oven at 45° C. overnight to give 60.0 g (101%) of (2S,3S)-3-(3-acetoxy-2-methyl-benzoylamino)-2-hydroxy-4-phenyl-butyric acid ethyl ester as an off white solid: ¹H NMR (300 MHz, CDCl₃) δ 6.93-7.25 (m, 8H), 6.02 (d, J=9.0 Hz, 1H), 4.78-4.87 (m, 1H), 4.37 (d, J=3.0 Hz, 1H), 4.13 (app dq, J=10.7, 7.2 Hz, 1H), 4.04 (app dq, J=10.7, 7.1 Hz, 1H), 2.79 (d, J=7.5 Hz, 2H), 2.24 (s, 3H), 1.95 (s, 3H), 1.21 (t, J=7.1 Hz, 3H); ¹³C NMR (75 MHz, DMSO-d₆) δ 172.7, 169.3, 168.1, 149.6, 139.5, 139.2, 129.5, 128.3, 128.0, 126.6, 126.4, 124.8, 123.4, 73.4, 60.7, 53.5, 35.5, 20.8, 14.5, 12.6; MS (Cl) m/z 400.1751 (400.1760 calcd for C₂₂H₂₆NO₆, M+H⁺); elemental analysis calcd for C₂₂H₂₅NO₆: C, 66.15; H, 6.31; N, 3.51; found: C, 65.90; H, 6.28; N, 3.39.

### Example 15

### Preparation of (2S,3S)-2-Hydroxy-3-(3-hydroxy-2-methyl-benzoylamino)₄-phenyl-butyric Acid

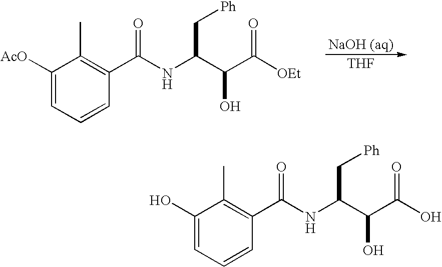

NaOH (108 mL of a 3 N aqueous solution, 324 mmol) was added to a suspension of (2S,3S)-3-(3-acetoxy-2-methyl-benzoylamino)-2-hydroxy-4-phenyl-butyric acid ethyl ester (58.9 g, 147 mmol) and tetrahydrofuran (300 mL) at ambient temperature. The resulting warm biphasic solution was stirred at ambient temperature overnight. The flask was equipped with a distillation head and was submerged in an oil bath. A total of 340 mL distillate was collected at one atmosphere with the oil bath temperature range of 75-125° C. The resulting clear yellow solution remaining in the boiling pot was diluted with H₂O (100 mL), and was then cooled to 0° C. 6 N HCl (60 mL) was slowly added, followed by ethyl acetate (250 mL), and the resulting mixture was warmed to ambient temperature with vigorous stirring. The resulting layers were separated. The organic fraction was washed with one-third saturated aqueous NaCl, and was then dried over MgSO₄, filtered, and was then concentrated to approximately 220 mL using a rotary evaporator. The resulting solution was allowed to stir at ambient temperature overnight. The resulting suspension of solid was vacuum-filtered, and the solid was rinsed with n-heptane (2×200 mL). After drying in a vacuum oven at 45° C. for 48 h, 45.1 g (92.8%) of (2S,3S)-2-hydroxy-3-(3-hydroxy-2-methyl-benzoylamino)₄-phenyl-butyric acid was obtained as a white solid: ¹H NMR (300 MHz, DMSO-d₆) δ 12.6 (br s, 1H), 9.35 (s, 1H), 8.05 (d, J=9.0 Hz, 1H), 7.16-7.30 (m, 5H), 6.95 (t, J=7.8 Hz, 1H), 6.77 (d, J=7.3 Hz, 1H), 6.53 (d, J=6.7 Hz, 1H), 5.63 (br s, 1H), 4.38-4.49 (m, 1H), 4.07 (d, J=5.9 Hz, 1H), 2.87 (app dd, J=3.0, 13.8 Hz, 1H), 2.74 (app dd, J=11.2, 13.9 Hz, 1H), 1.80 (s, 3H); ¹³C NMR (75 MHz, DMSO-d₆) δ 174.2, 168.9, 155.4, 139.3, 139.2, 129.3, 128.1, 126.0, 125.8, 121.5, 117.6, 115.2, 73.0, 53.1, 35.1, 12.3; MS (Cl) m/z 330.1348 (330.1341 calcd for C₁₈H₂₀NO₅, M+H⁺).

### Example 16

### Preparation of (4R)-3-[(2S,3S)-2-Hydroxy-3-(3-hydroxy-2-methyl-benzoylamino)-4-phenyl-butyryl]-5,5-dimethyl-thiazolidine-4-carboxylic acid 2-methyl-benzylamide

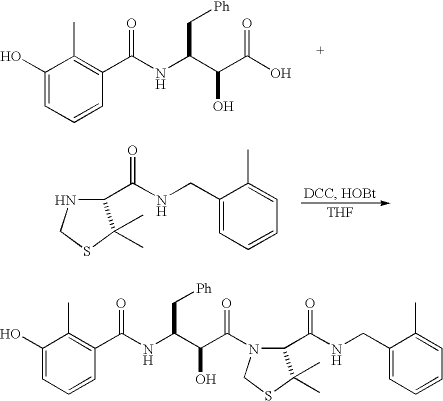

A solution of dicyclohexylcarbodiimide (3.29 g, 15.9 mmol) and tetrahydrofuran (15 mL) was slowly added to a solution of (2S,3S)-2-hydroxy-3-(3-hydroxy-2-methyl-benzoylamino)₄-phenyl-butyric acid (5.00 g, 15.2 mmol), (4R)-5,5-dimethyl-thiazolidine-4-carboxylic acid 2-methyl-benzylamide (4.21 g, 15.9 mmol) (which can be prepared according to the procedure found in H. Hayashi, et al. J. Med. Chem. 1999, 42, 1789; R. Kato et al., U.S. Pat. No. 5,932,550; and J. R. Tata et al., PCT Publication No. WO 01/05230 A1) HOBt.H₂O (2.05 g, 15.2 mmol), and tetrahydrofuran (50 mL) at ambient temperature. The resulting suspension was stirred at ambient temperature overnight. Ethyl acetate (35 mL) was added and the suspension was then vacuum-filtered, using ethyl acetate (20 mL) for rinsing. The filtrate was sequentially washed with 5% aqueous Na₂CO₃ (50 mL), 0.5 N HCl (50 mL), and one-quarter saturated aqueous NaCl (50 mL). The resulting organic fraction was then dried over MgSO₄, filtered, and then concentrated to a volume of 45 mL using a rotary evaporator. The solution was allowed to stir at ambient temperature overnight. The resulting suspension was vacuum-filtered, and the solid was discarded. The filtrate was concentrated with a rotary evaporator to give crude (4R)-3-[(2S,3S)-2-hydroxy-3-(3-hydroxy-2-methyl-benzoylamino)-4-phenyl-butyryl]-5,5-dimethyl-thiazolidine-4-carboxylic acid 2-methyl-benzylamide as a yellowish solid. This solid was dissolved in isopropyl acetate (62 mL) and the crystallizing mixture was stirred at ambient temperature overnight. The suspension was vacuum-filtered. The solid was rinsed with isopropyl acetate (2×20 mL), and was then dried in a vacuum oven at 45° C. for 24 h to give 5.60 g (64.1%) of (4R)-3-[(2S,3S)-2-hydroxy-3-(3-hydroxy-2-methyl-benzoylamino)-4-phenyl-butyryl]-5,5-dimethyl-thiazolidine-4-carboxylic acid 2-methyl-benzylamide as a white solid: ¹H NMR (300 MHz, DMSO-d₆) displayed a ˜7:1 mixture of rotamers, major rotamer resonances δ 9.37 (s, 1H), 8.32 (t, J=5.6 Hz, 1H), 8.14 (d, J=8.3 Hz, 1H), 7.10-7.34 (m, 9H), 6.95 (t, J=7.7 Hz, 1H), 6.78 (d, J=7.7 Hz, 1H), 6.56 (d, J=7.1 Hz, 1H), 5.46 (br s, 1H), 5.08 (ABq, J_{AB}=9.1 Hz, 2H), 4.38-4.50 (m, 3H), 4.11 (dd, J=4.7, 15.1 Hz, 1H), 2.85 (app dd, J=2.8, 13.6 Hz, 1H), 2.73 (app dd, J=10.5, 13.5 Hz, 1H), 2.27 (s, 3H), 1.84 (s, 3H), 1.50 (s, 3H), 1.36 (s, 3H) [characteristic minor rotamer resonances δ 8.19 (d, J=8.5 Hz), 8.07 (t, J=5.7 Hz), 6.49 (d, J=7.5 Hz), 4.93 (s), 4.80 (ABq, J_{AB}=9.7 Hz), 1.82 (s), 1.40 (s)]; ¹³C NMR (75 MHz, DMSO-d₆) displayed a ˜7:1 mixture of rotamers, major rotamer resonances δ 170.5, 169.5, 168.3, 155.7, 139.7, 139.4, 137.1, 136.0, 130.2, 129.9, 128.3, 128.2, 127.2, 126.2, 126.1, 126.0, 121.8, 117.8, 115.6, 72.4, 71.9, 53.2, 51.5, 48.1, 40.8, 34.2, 30.6, 25.0, 19.1, 12.6 [characteristic minor rotamer resonances δ 171.4, 169.6, 168.8, 139.0, 137.0, 135.8, 129.5, 128.4, 71.9, 54.3, 31.5, 24.8]; MS (Cl) m/z 576.2552 (576.2532 calcd for C₃₂H₃₈N₃O₅S, M+H⁺).

### Example 17

### Preparation of (4R)-3-[(2S,3S)-2-Hydroxy-3-(3-hydroxy-2-methyl-benzoylamino)-4-phenyl-butyryl]-5,5-dimethyl-thiazolidine-4-carboxylic acid 2-methyl-benzylamide

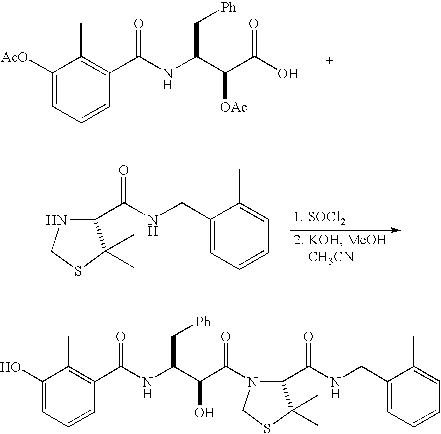

A solution of (2S,3S)-2-acetoxy-3-(3-acetoxy-2-methyl-benzoylamino)-4-phenyl-butyric acid (140 kg, 339 mol), CH₃CN (560 L), and pyridine (64.3 kg, 813 mol) is cooled to 15° C. SOCl₂ (44.3 kg, 373 mol) is charged while maintaining the temperature at 15° C. The mixture is held at 15° C. for 1 h. A separate reactor is charged with (4R)-5,5-dimethyl-thiazolidine-4-carboxylic acid 2-methyl-benzylamide (89.7 kg, 339 mol), CH₃CN (254 L), and pyridine (29.5 kg, 373 mol), and is then cooled to 15° C. The (2S,3S)-2-acetoxy-3-(3-acetoxy-2-methyl-benzoylamino)₄-phenyl-butyric acid chloride solution is added to the (4R)-5,5-dimethyl-thiazolidine-4-carboxylic acid 2-methyl-benzylamide solution, while maintaining the temperature at 15° C. The mixture is held at 15° C. for 6 h. A separate reactor is charged with KOH (167 kg, 2709 mol) and methanol (280 L) using a 0° C. cooling jacket. The resulting KOH/methanol solution is cooled to 5° C. The crude acetic acid ester mixture is added to the KOH/methanol solution while maintaining the temperature at 10° C. After addition is complete, the mixture is held at 25° C. for 3 h. The mixture is charged with H₂O (840 L) and ethyl acetate (840 L), and is then followed by acidification to pH 54.5 with concentrated HCl (85 kg) while maintaining the temperature at 20° C. The resulting layers are separated. The organic fraction is sequentially washed with 6.8 wt. % aqueous NaHCO₃ (770 L), an aqueous HCl/NaCl solution (H₂O: 875 L; conc. HCl: 207 kg; NaCl: 56 kg), 8.5 wt. % aqueous NaHCO₃ (322 L), and then with 3.8 wt. % aqueous NaCl (728 L). The resulting organic fraction is partially concentrated by distillation at one atmosphere. The solvent is exchanged with ethyl acetate by continuing distillation and maintaining the pot temperature at ≧70° C. Ethyl acetate is added such that the pot volume remained at approximately 840 L. The solution is then cooled to 20° C. and held at this temperature until crystallization is observed. n-Heptane (280 L) is added and the suspension is agitated at 15° C. for 4 h. The crystals are rinsed using cold 2.4:1 (v/v) ethyl acetate/n-heptane for rinsing. The wet cake is dried under vacuum at 45° C. to provide (4R)-3-[(2S,3S)-2-hydroxy-3-(3-hydroxy-2-methyl-benzoylamino)₄-phenyl-butyryl]-5,5-dimethyl-thiazolidine-4-carboxylic acid 2-methyl-benzylamide.

1. A method of preparing a compound of formula (I), or a salt or solvate thereof: 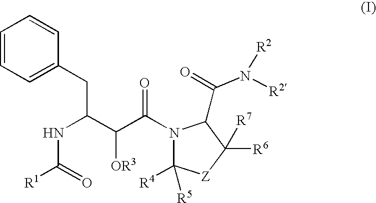
wherein:
R¹ is phenyl optionally substituted by at least one substituent independently chosen from C₁-C₆ alkyl, hydroxyl, C₁-C₆ alkylcarbonyloxy, C₆-C₁₀ arylcarbonyloxy, and heteroarylcarbonyloxy;
R² is C₂-C₆ alkenyl, C₁-C₆ alkyl optionally substituted with at least one halogen, or —(CR⁴R⁵)ₙR⁸;
n is an integer from 0 to 5;
R^{2′} is H or C₁-C₄ alkyl;
Z is S, O, SO, SO₂, CH₂, or CFH;
R³ is hydrogen or a hydroxyl protecting group;
each R⁴, R⁵, R⁶ and R⁷ are independently selected from H and C₁-C₆ alkyl; and
R⁸ is C₆-C₁₀ aryl optionally substituted at least one substituent selected from C₁-C₆ alkyl, hydroxyl, and halogen;
comprising:
reacting a compound of formula (II), wherein Y¹ is hydroxyl or a leaving group and R¹ is as described for formula (I), with a compound of formula (III), or a salt or solvate thereof,

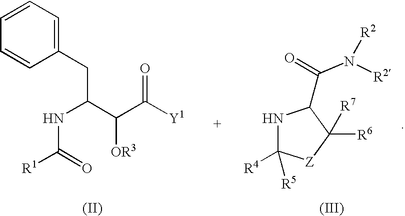

2. A method according to claim 1, wherein in the compound of formula (II), Y¹ is hydroxyl.

3. A method according to claim 1, wherein in the compound of formula (I):
n is 0, 1, 2, or 3;
R^{2′} is H;
Z is S, O, CH₂, or CFH;
R⁴ and R⁵ are hydrogen; and
R⁶ and R⁷ are C₁-C₆ alkyl.

4. A method according to claim 3, wherein in the compound of formula (I):
Z is S;
R³ is hydrogen; and
R⁶ and R⁷ are methyl.

5. A method according to claim 3, wherein in the compound of formula (I):
Z is S;
R³ is a hydroxyl protecting group;
R⁴ and R⁵ are hydrogen;
R⁶ and R⁷ are methyl; and
R⁸ is phenyl optionally substituted with at least one substituent selected from C₁-C₆ alkyl, hydroxyl, and halogen.

6. A method according to claim 3, wherein in the compound of formula (I):
R¹ is phenyl optionally substituted by at least one substituent independently chosen from methyl, hydroxyl, and methylcarbonyloxy;
R² is C₂-C₆ alkenyl, C₁-C₆ alkyl optionally substituted with at least one halogen, or —CH₂R⁸;
Z is S;
R⁵ and R⁷ are methyl; and
R⁸ is phenyl substituted with at least one methyl.

7. A method according to claim 6, wherein in the compound of formula (I) R² is C₂-C₆ alkenyl.

8. A method according to claim 7, wherein in the compound of formula (1):
R¹ is phenyl substituted by methyl and hydroxyl;
R² is allyl; and
R³ is hydrogen or methylcarbonyl.

9. A method according to claim 7, wherein in the compound of formula (1):
R¹ is phenyl substituted with methyl and methylcarbonyloxy;
R² is allyl; and
R³ is methylcarbonyl.

10. A method according to claim 8, wherein the compound of formula (1) is:

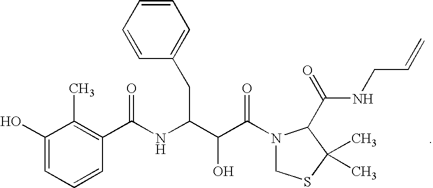

11. A method of preparing a compound of formula (I-A), 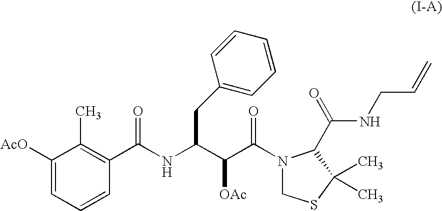
comprising:
reacting a compound of formula (II-A) with a compound of formula (III-A), or a salt or solvate thereof,

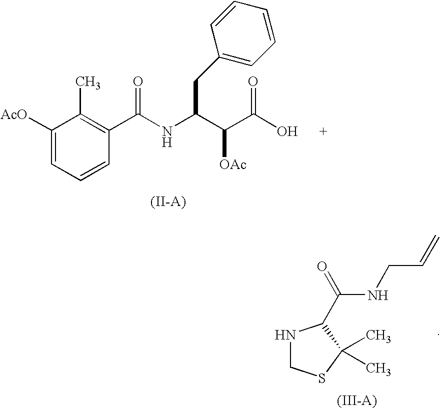

12. A method for preparing a compound of formula (I-B), 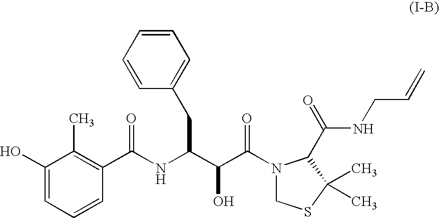
comprising:
(i) reacting a compound of formula (II-A) with a compound of formula (III-A), or a salt or solvate thereof, 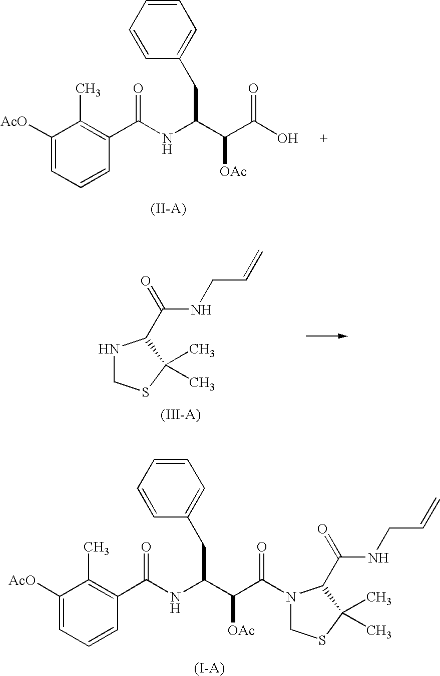
to afford a compound of formula (I-A); and
(ii) deprotecting the compound of formula (I-A).

13. A compound of formula (I-A),

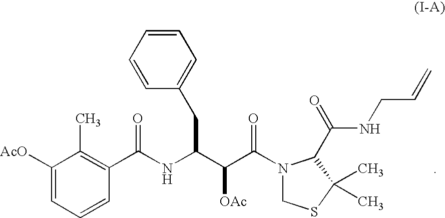

14. A compound of formula (II-A), [chemical structure]
